(19) United States
(12) Patent Application Publication
(10) Pub. No.: US 2010/0292289 A1
(43) Pub. Date: Nov. 18, 2010

(54) TREATMENT OF METABOLIC SYNDROME WITH NOVEL AMIDES

(75) Inventor: James R. Hauske, La Jolla, CA (US)

Correspondence Address: ROPES & GRAY LLP, PATENT DOCKETING 39/41, ONE INTERNATIONAL PLACE, BOSTON, MA, 02110-2624, US

(73) Assignee: AMPLA Pharmaceuticals Inc., La Jolla, CA (US)

(21) Appl. No.: 12/745,339

(22) PCT Filed: Nov. 26, 2008
(86) PCT No.: PCT/US08/13168

§ 371 Date: Jun. 24, 2010

Related U.S. Application Data
(60) Provisional application No. 61/005,043, filed on Nov. 30, 2007.
(60) Provisional application No. 61/070,503, filed on Mar. 24, 2008.
(60) Provisional application No. 61/124,204, filed on Apr. 15, 2008.

(51) Int. Cl. A61K 31/165; C07C 235/78; C07D 209/26; A61K 31/405; C07D 487/04; A61K 31/407; A61P 3/04; A61P 25/24

(52) U.S. Cl. 514/413; 564/169; 514/621; 548/500; 514/415; 548/453

(57) ABSTRACT

The present invention relates to the treatment of metabolic syndrome or disorders associated with metabolic syndrome comprising administering a compound of the invention.

### RELATED APPLICATIONS

This application claims priority to U.S. provisional application Ser. No. 61/005,043, filed Nov. 30, 2007, U.S. provisional application Ser. No. 61/070,503, filed Mar. 24, 2008, and U.S. provisional application Ser. No. 61/124,204, filed Apr. 15, 2008. The disclosures of the foregoing applications are hereby incorporated by reference in their entirety.

### BACKGROUND

### Obesity

According to the National Health and Nutrition Examination Survey (NHANES III, 1988 to 1994), between one third and one half of men and women in the United States are overweight. In the United States, sixty percent of men and fifty-one percent of women, of the age of 20 or older, are either overweight or obese. In addition, a large percentage of children in the United States are overweight or obese.

Obesity is a condition of complex origin. Increasing evidence suggests that obesity is not a simple problem of self-control but is a complex disorder involving appetite regulation and energy metabolism. In addition, obesity is associated with a variety of conditions associated with increased morbidity and mortality in a population. Although the etiology of obesity is not definitively established, genetic, metabolic, biochemical, cultural and psychosocial factors are believed to contribute. In general, obesity has been described as a condition in which excess body fat puts an individual at a health risk.

There is strong evidence that obesity is associated with increased morbidity and mortality. Disease risk, such as cardiovascular disease risk and type 2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (Kenchaiah et al., N. Engl. J. Med. 347:305, 2002; Massie, N. Engl. J. Med 347:358, 2002). In addition, there is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia.

Although diet and exercise provide a simple process to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients.

It is not only obese subjects who wish to lose weight. People with weight within the recommended range, for example, in the upper part of the recommended range, may wish to reduce their weight, to bring it closer to the ideal weight. Thus, a need remains for agents that can be used to effect weight loss in overweight and obese subjects.

### Metabolic Syndrome

Metabolic syndrome (also known as “syndrome X,” “dysmetabolic syndrome,” “obesity syndrome,” and “Reaven's syndrome”) has emerged as a growing problem. For example, metabolic syndrome has become increasingly common in the United States. It is estimated that about 47 million adults in the United States have the syndrome.

Metabolic syndrome is generally a constellation of metabolic disorders that all result from, or are associated with, a primary disorder of insulin resistance. Accordingly, the syndrome is sometimes referred to as “insulin resistance syndrome.” Insulin resistance is characterized by disorders in which the body cannot use insulin efficiently and the body's tissues do not respond normally to insulin. As a result, insulin levels become elevated in the body's attempt to overcome the resistance to insulin. The elevated insulin levels lead, directly or indirectly, to the other metabolic abnormalities.

Some people are genetically predisposed to insulin resistance, while other people acquire factors that lead to insulin resistance. Acquired factors, such as excess body fat and physical inactivity, can elicit insulin resistance, and more broadly, clinical metabolic syndrome. Because of this relationship between insulin resistance and metabolic syndrome, it is believed that the underlying causes of this syndrome are obesity, physical inactivity and genetic factors. In fact, most people with insulin resistance and metabolic syndrome have central obesity (excessive fat tissue in and around the abdomen). The biologic mechanisms at the molecular level between insulin resistance and metabolic risk factors are not yet fully understood and appear to be complex.

Metabolic syndrome is typically characterized by a group of metabolic risk factors that include 1) central obesity; 2) atherogenic dyslipidemia (blood fat disorders comprising mainly high triglycerides (“TG”) and low HDL-cholesterol (interchangeably referred to herein as “HDL”) that foster plaque buildups in artery walls); 3) raised blood pressure; 4) insulin resistance or glucose intolerance (the body can't properly use insulin or blood sugar); 5) prothrombotic state (e.g., high fibrinogen or plasminogen activator inhibitor in the blood); and 6) a proinflammatory state (e.g., elevated high-sensitivity C-reactive protein in the blood). The National Cholesterol Education Program (NCEP) Adult Treatment Panel (ATP) Ill guidelines define metabolic syndrome by the following five clinical parameters: a) a waist circumference greater than 102 cm for men, and greater than 88 cm for women; b) a triglyceride level greater than 150 mg/dl; c) an HDL-cholesterol less than 40 mg/dl for men, and less than 50 mg/dl for women; d) a blood pressure greater than or equal to 130/85 mmHg; and e) a fasting glucose greater than 110 mg/dl.

According to the American Heart Association, however, there are no well-accepted criteria for diagnosing metabolic syndrome. Some guidelines suggest that metabolic syndrome involves four general factors: obesity; diabetes; hypertension; and high lipids. According to the NCEP ATP III guidelines above, the presence of at least three of these factors meets the medical diagnosis of metabolic syndrome.

Although there is no complete agreement on the individual risk or prevalence of each factor, it is known that the syndrome, as generally agreed upon by those skilled in the field, poses a significant health risk to individuals. A person having one factor associated with the syndrome has an increased risk for having one or more of the others. The more factors that are present, the greater the risks to the person's health. When the factors are present as a group, i.e., metabolic syndrome, the risk for cardiovascular disease and premature death is very high.

For example, a person with the metabolic syndrome is at an increased risk of coronary heart disease, other diseases related to plaque buildups in artery walls (e.g., stroke and peripheral vascular disease), prostate cancer, and type 2 diabetes. It is also known that when diabetes occurs, the high risk of cardiovascular complications increases.

Generally, patients suffering from the syndrome are prescribed a change in lifestyle, e.g., an increase in exercise and a change to a healthy diet. The goal of exercise and diet programs is to reduce body weight to within 20% of the “ideal” body weight calculated for age and height.

In some cases, diet and exercise regimens are supplemented with treatments for lipid abnormalities, clotting disorders, and hypertension. For example, patients with the syndrome typically have several disorders of coagulation that make it easier to form blood clots within blood vessels. These blood clots are often a precipitating factor in developing heart attacks. Patients with the syndrome are often placed on daily aspirin therapy to specifically help prevent such clotting events. Furthermore, high blood pressure is present in more than half the people with the syndrome, and in the setting of insulin resistance, high blood pressure is especially important as a risk factor. Some studies have suggested that successfully treating hypertension in patients with diabetes can reduce the risk of death and heart disease by a substantial amount. Additionally, patients have been treated to specifically reduce LDL-cholesterol (interchangeably referred to herein as “LDL”) levels, reduce triglyceride levels, and raise HDL levels. Given the increasing prevalence of this syndrome, there remains a need for additional and effective treatments of the syndrome.

### SUMMARY OF INVENTION

The present invention provides novel compounds, including purified preparations of those compounds. For instance, the invention provides compounds of formula I or a pharmaceutically acceptable salt thereof, or a solvate of the compound or its salt:

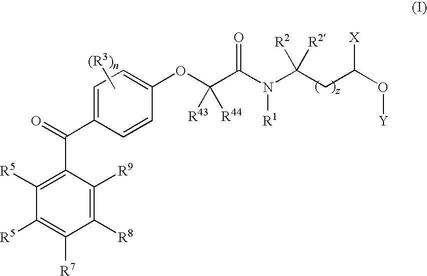

wherein:
X represents H, methyl, or an optionally substituted aryl or heteroaryl ring system;
Y represents an optionally substituted aryl or heteroaryl ring system;
n represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³ independently for each occurrence, represents a substituent;
R⁵, R⁶, R⁷, R⁸, and R⁹ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system.

The invention further provides compounds of formula Ia or a pharmaceutically acceptable salt thereof, or a solvate of the compound or its salt:

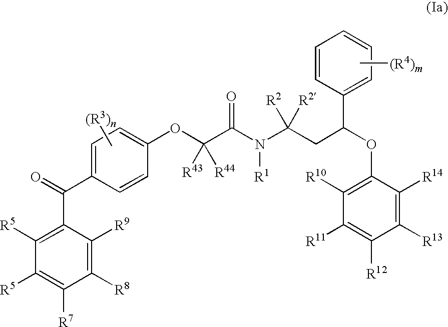

wherein:
n-represents an integer from 0 to 4;
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³ and R⁴, each independently for each occurrence, represent a substituent;
R⁵, R⁶, R⁷, R⁸, R⁹, R¹⁰, R¹¹, R¹², R¹³, and R¹⁴ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system.

The invention further provides compounds of formula II or a pharmaceutically acceptable salt thereof, or a solvate of the compound or its salt:

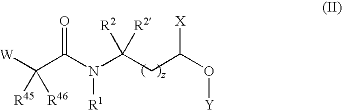

wherein:
X represents H, methyl, or an optionally substituted aryl or heteroaryl ring system;
Y represents an optionally substituted aryl or heteroaryl ring system;
W represents

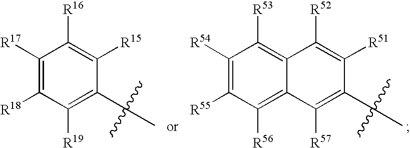

z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R¹⁵, R¹⁶, R¹⁷, R¹⁸, R¹⁹, R⁵¹, R⁵², R⁵³, R⁵⁴, R⁵⁵, R⁵⁶ and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system.

In certain embodiments, the compound of formula II is represented by formula IIa or a pharmaceutically acceptable salt thereof, or a solvate of the compound or its salt:

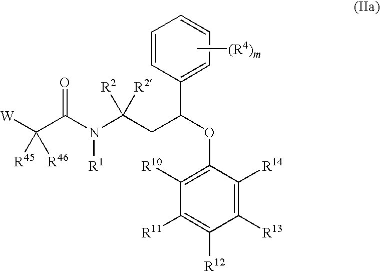

wherein:
W represents

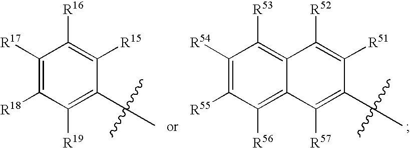

m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent;
R¹⁰, R¹¹, R¹², R¹³, R¹⁴, R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system.

The invention further provides compounds of formula III or a pharmaceutically acceptable salt thereof, or a solvate of the compound or its salt:

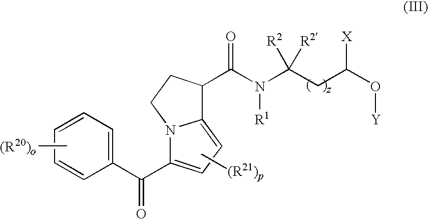

wherein:
X represents H or an optionally substituted aryl or heteroaryl ring system;
Y represents an optionally substituted aryl or heteroaryl ring system;
o represents an integer from 0 to 5;
p represents an integer from 0 to 2;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system; and
R²⁰ and R²¹ each independently for each occurrence represent a substituent.

In certain embodiments, the compound of formula III is represented by formula Ma or a pharmaceutically acceptable salt thereof, or a solvate of the compound or its salt:

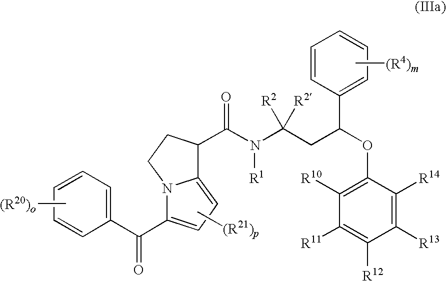

wherein:
m represents an integer from 0 to 5;
o represents an integer from 0 to 5;
p represents an integer from 0 to 2;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²⁰, and R²¹, each independently for each occurrence, represents a substituent; and
R¹⁰, R¹¹, R¹², R¹³, and R¹⁴ each independently represent hydrogen or a substituent.

The invention further provides compounds of formula IV or a pharmaceutically acceptable salt thereof, or a solvate of the compound or its salt:

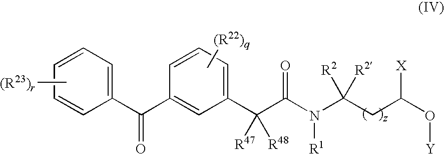

wherein:
X represents H or an optionally substituted aryl or heteroaryl ring system;
Y represents an optionally substituted aryl or heteroaryl ring system;
r represents an integer from 0 to 5;
q represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²² and R²³ each independently for each occurrence represent a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system.

In certain embodiments, the compound of formula IV is represented by formula IVa or a pharmaceutically acceptable salt thereof, or a solvate of the compound or its salt:

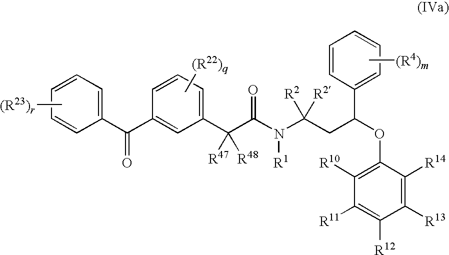

wherein:
m represents an integer from 0 to 5;
r represents an integer from 0 to 5;
q represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²², and R²³, each independently for each occurrence, represents a substituent;
R¹⁰, R¹¹, R¹², R¹³, and R¹⁴ each independently represent hydrogen or a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system.

The invention further provides compounds of formula V or a pharmaceutically acceptable salt thereof, or a solvate of the compound or its salt:

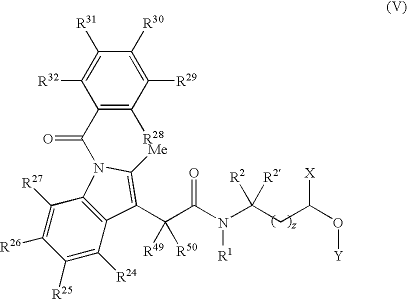

wherein:
X represents H or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system.

In certain embodiments, the compound of formula V is represented by formula Va or a pharmaceutically acceptable salt thereof, or a solvate of the compound or its salt:

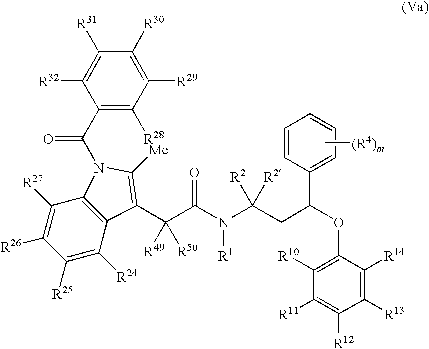

wherein:
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent; and
R¹⁰, R¹¹, R¹², R¹³, R¹⁴, R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system.

The invention further provides compounds of formula VI or a pharmaceutically acceptable salt thereof, or a solvate of the compound or its salt:

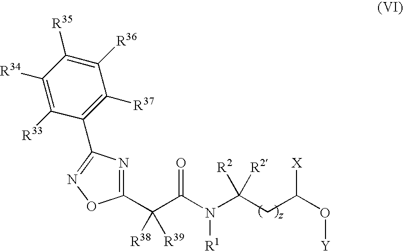

wherein:
X represents H, methyl, or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³³, R³⁴, R³⁵, R³⁶, and R³⁷ each independently represent hydrogen or a substituent; and
R³⁸ and R³⁹ each independently represent hydrogen or a substituent, or R³⁸ and R³⁹ taken together with the carbon to which they are attached form a three- to six-membered cyclic ring system.

In another aspect, the present invention provides a method of treating obesity, metabolic syndrome or a disorder associated with metabolic syndrome (e.g., obesity, diabetes, hypertension, and hyperlipidemia) in a mammal comprising administering to a mammal suffering from obesity, metabolic syndrome or a disorder associated with metabolic syndrome (e.g., obesity, diabetes, hypertension, and hyperlipidemia) a compound of the invention (e.g., a compound of any of formulae I, Ia, II, IIa, III, IIIa, IV, IVa, V, Va, or VI).

In certain embodiments, the disorder associated with metabolic syndrome is diabetes.

In another aspect, the present invention provides a method of treating depression in a mammal comprising administering to a mammal suffering from depression a compound of the invention (e.g., a compound of any of formulae I, Ia, II, IIa, III, IIIa, IV, IVa, V, Va, or VI).

In preferred embodiments of the methods of the invention, the mammal is a human.

### DETAILED DESCRIPTION OF THE DRAWINGS

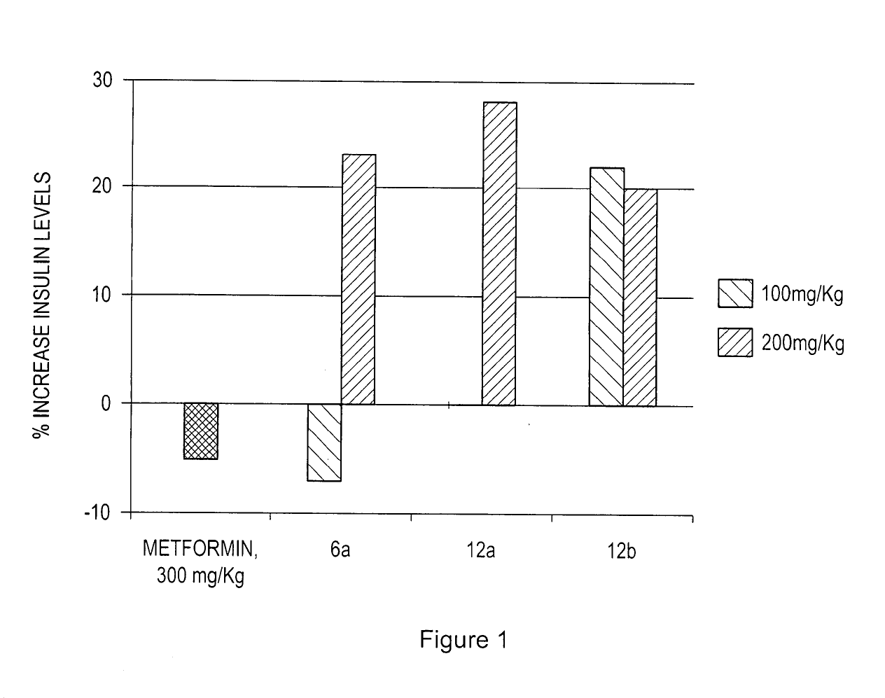

FIG. 1 shows the percentage increase in insulin levels after oral administration of test compounds at 100 and 200 mg/kg and metformin at 300 mg/kg.

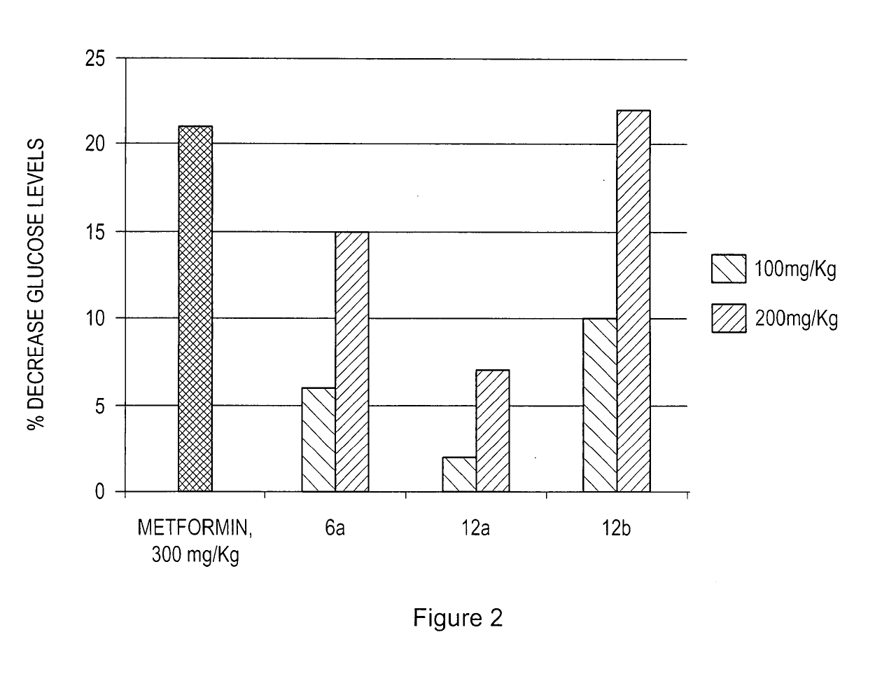

FIG. 2 shows the percentage decrease in glucose levels after oral administration of test compounds at 100 and 200 mg/kg and metformin at 300 mg/kg.

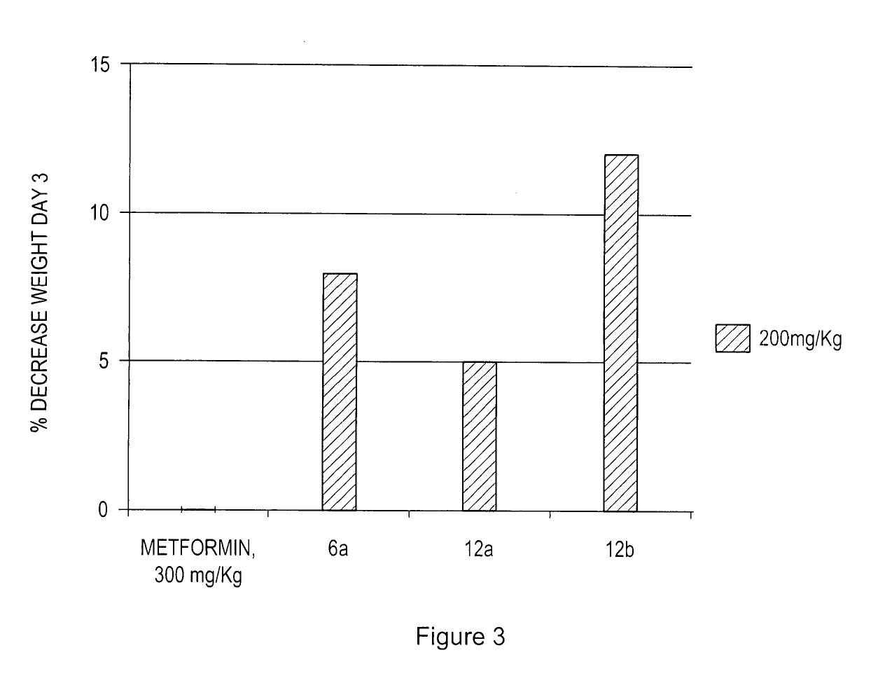

FIG. 3 shows the percentage decrease in weight after oral administration of test compounds at 200 mg/kg and metformin at 300 mg/kg.

### DETAILED DESCRIPTION OF THE INVENTION

The present invention provides certain novel compounds, including purified preparations of those compounds. For instance, the invention provides compounds of formula I or a pharmaceutically acceptable salt thereof, or a solvate of the compound or its salt:

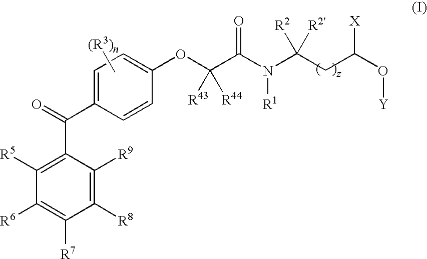

wherein:
X represents H, methyl, or an optionally substituted aryl or heteroaryl ring system;
Y represents an optionally substituted aryl or heteroaryl ring system;
n represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³ independently for each occurrence, represents a substituent;
R⁵, R⁶, R⁷, R⁸, and R⁹ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R⁵, R⁶, R⁷, R⁸, and R⁹ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R⁵, R⁶, R⁸, and R⁹ are hydrogen, and R⁷ is halogen. In certain such embodiments, R⁷ is chloro.

In certain embodiments, R³ independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, n is 0.

In certain embodiments, X represents an optionally substituted aryl or heteroaryl ring system.

In certain embodiments, R⁴³ and R⁴⁴ are each optionally substituted lower alkyl, such as methyl. In certain embodiments, R⁴³ and R⁴⁴ taken together with the carbon to which they are attached form a cyclopropane or cyclobutane ring.

In certain embodiments, the compound is represented by formula Ia or a pharmaceutically acceptable salt thereof, or a solvate of the compound or its salt:

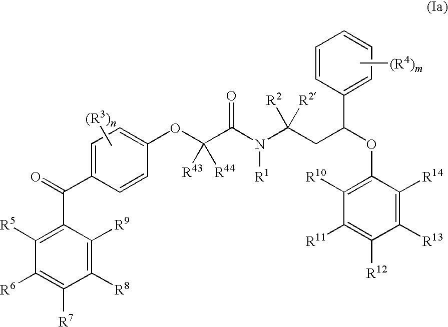

wherein:
n represents an integer from 0 to 4;
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system; R³ and R⁴, each independently for each occurrence, represent a substituent;
R⁵, R⁶, R⁷, R⁸, R⁹, R¹⁰, R¹¹, R¹², R¹³, and R¹⁴ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system.

In certain embodiments, R⁵, R⁶, R⁷, R⁸, R⁹, R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R⁵, R⁶, R⁸, R⁹, R¹⁰, R¹¹, R¹³, and R¹⁴ are hydrogen, R⁷ is halogen, and R¹² is optionally substituted lower alkyl. In certain such embodiments, R⁷ is chloro and R¹² is trifluoromethyl.

In certain embodiments, R³ and R⁴ each independently for each occurrence represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, m is 0.

In certain embodiments, n is 0.

In certain embodiments, R⁴³ and R⁴⁴ are each optionally substituted lower alkyl, such as methyl. In certain embodiments, R⁴³ and R⁴⁴ taken together with the carbon to which they are attached form a cyclopropane or cyclobutane ring.

In certain embodiments, a compound of formula Ia has the structure 1:

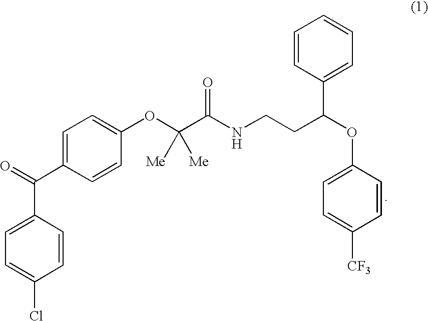

In certain embodiments, the compound of formula Ia has the structure 1 and is enriched for the (R) enantiomer (e.g., compound Ia,

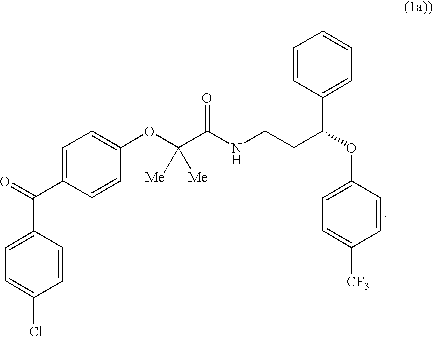

In certain such embodiments, the compound is substantially free of the (S) enantiomer.

Further exemplary compounds of formula Ia include the following:

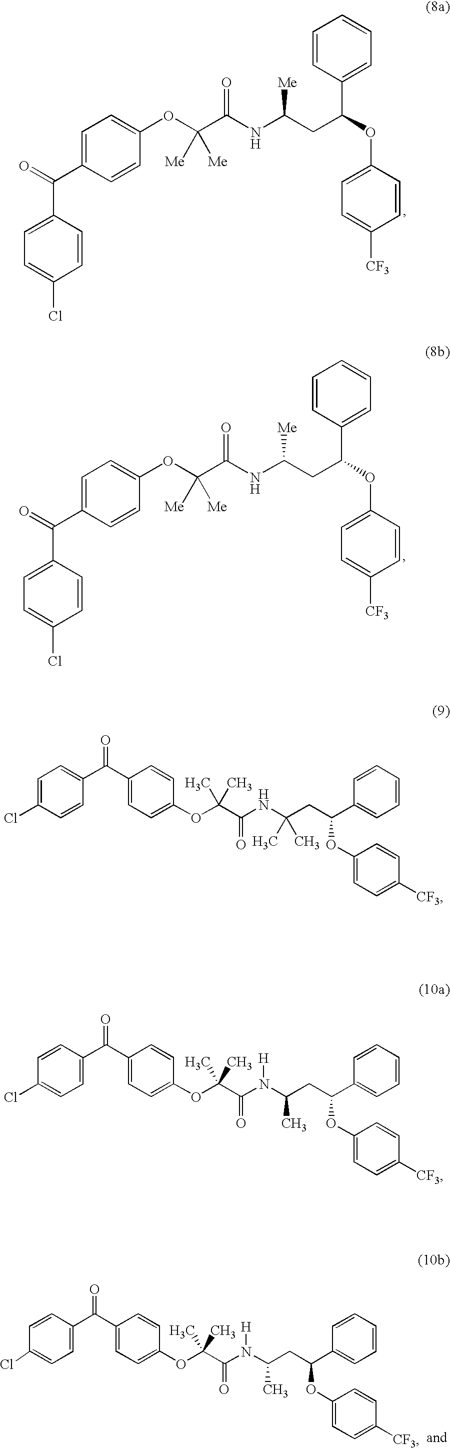

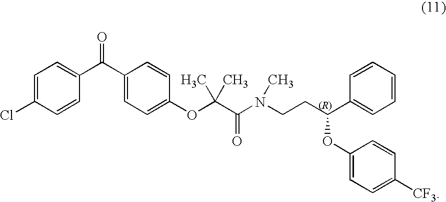

The invention further provides compounds of formula II or a pharmaceutically acceptable salt thereof, or a solvate of the compound or its salt:

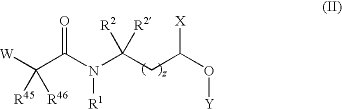

wherein:
X represents H, methyl, or an optionally substituted aryl or heteroaryl ring system;
Y represents an optionally substituted aryl or heteroaryl ring system;
W represents

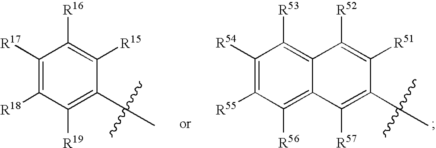

z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system.

In certain embodiments, X represents an optionally substituted aryl or heteroaryl ring system. In certain embodiments, X represents H or an optionally substituted aryl or heteroaryl ring system. In certain embodiments, X represents methyl or an optionally substituted aryl or heteroaryl ring system.

In certain embodiments wherein X is methyl, z is 1 or 2.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹⁵, R¹⁶, R¹⁷, R¹⁸, and R¹⁹ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, such as an optionally substituted tertiary aminoalkyl, optionally substituted amido, optionally substituted acylamino, optionally substituted carbocyclylalkyl, optionally substituted heterocyclyl, such as an optionally substituted nitrogen-containing heterocyclyl (e.g., morpholine, piperidine, piperazine, or pyrrolidine), optionally substituted heterocyclylalkyl, such as an optionally substituted nitrogen-containing heterocyclylalkyl, optionally substituted aryl, cyano, or nitro. In certain embodiments, R¹⁵, R¹⁶, R¹⁸, and R¹⁹ are hydrogen, and R¹⁷ is optionally substituted lower alkyl. In certain such embodiments, R¹⁷ is isobutyl.

In certain embodiments, R⁵¹, R⁵², R⁵³, R⁵⁴, R⁵⁵, R⁵⁶, and R⁵⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, such as an optionally substituted tertiary aminoalkyl, optionally substituted amido, optionally substituted acylamino, optionally substituted carbocyclylalkyl, optionally substituted heterocyclyl, such as an optionally substituted nitrogen-containing heterocyclyl (e.g., morpholine, piperidine, piperazine, or pyrrolidine), optionally substituted heterocyclylalkyl, such as an optionally substituted nitrogen-containing heterocyclylalkyl, optionally substituted aryl, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, R⁴⁵ is hydrogen and R⁴⁶ is an optionally substituted lower alkyl, such as methyl. In certain embodiments, R⁴⁵ and R⁴⁶ taken together with the carbon to which they are attached form a cyclopropane or cyclobutane ring.

In certain embodiments, the compound of formula II is represented by formula IIa or a pharmaceutically acceptable salt thereof, or a solvate of the compound or its salt:

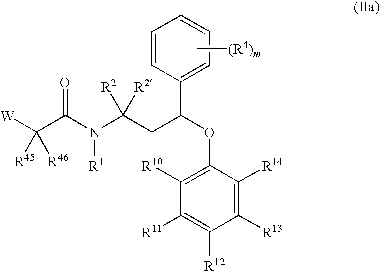

wherein:
W represents

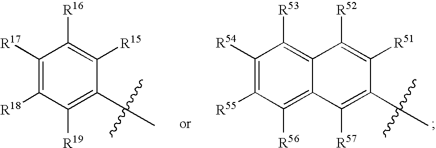

m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent;
R¹⁰, R¹¹, R¹², R¹³, R¹⁴, R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system.

In certain embodiments, R¹⁰, R¹¹, R¹², R¹³, and R¹⁴, each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, such as an optionally substituted tertiary aminoalkyl, optionally substituted amido, optionally substituted acylamino, optionally substituted heterocyclyl, such as an optionally substituted nitrogen-containing heterocyclyl (e.g., morpholine, piperidine, piperazine, or pyrrolidine), optionally substituted heterocyclylalkyl, such as an optionally substituted nitrogen-containing heterocyclylalkyl, cyano, or nitro. In certain embodiments, R¹⁰, R¹¹, R¹³, and R¹⁴ are hydrogen, and R¹² is optionally substituted lower alkyl. In certain such embodiments, R¹² is trifluoromethyl.
In certain embodiments, R¹⁵, R¹⁶, R¹⁷, R¹⁸, R¹⁹, R⁵¹, R⁵², R⁵³, R⁵⁴, R⁵⁵, R⁵⁶, and R⁵⁷, each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, such as an optionally substituted tertiary aminoalkyl, optionally substituted amido, optionally substituted acylamino, optionally substituted carbocyclylalkyl, optionally substituted heterocyclyl, such as an optionally substituted nitrogen-containing heterocyclyl (e.g., morpholine, piperidine, piperazine, or pyrrolidine), optionally substituted heterocyclylalkyl, such as an optionally substituted nitrogen-containing heterocyclylalkyl, optionally substituted aryl, cyano, or nitro. In certain embodiments, R¹⁵, R¹⁶, R¹⁸, and R¹⁹ are hydrogen, and R¹⁷ is optionally substituted lower alkyl. In certain such embodiments, R¹⁷ is isobutyl.

In certain embodiments, R⁴ independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, m is 0.

In certain embodiments, R⁴⁵ is hydrogen and R⁴⁶ is an optionally substituted lower alkyl, such as methyl. In certain embodiments, R⁴⁵ and R⁴⁶ taken together with the carbon to which they are attached form a cyclopropane or cyclobutane ring.

In certain embodiments, a compound of formula IIa has the structure 4:

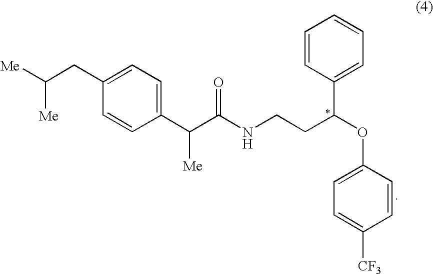

In certain embodiments, the compound of formula Ha has the structure 4 and is enriched for the (R) configuration at the indicated position (*). In certain such embodiments, the compound is substantially free of the (S) configuration at the indicated position (*).

Further exemplary compounds of formula II or IIa include:

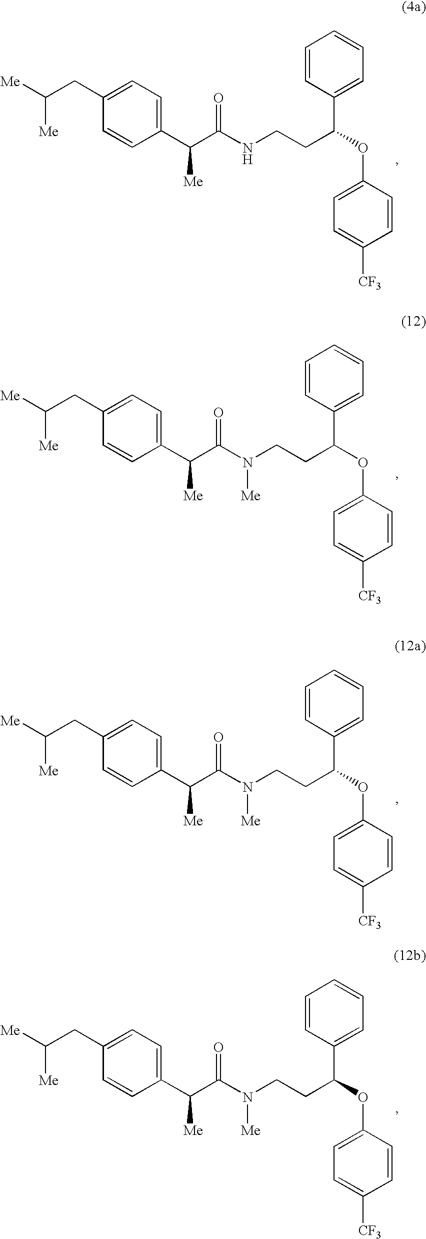

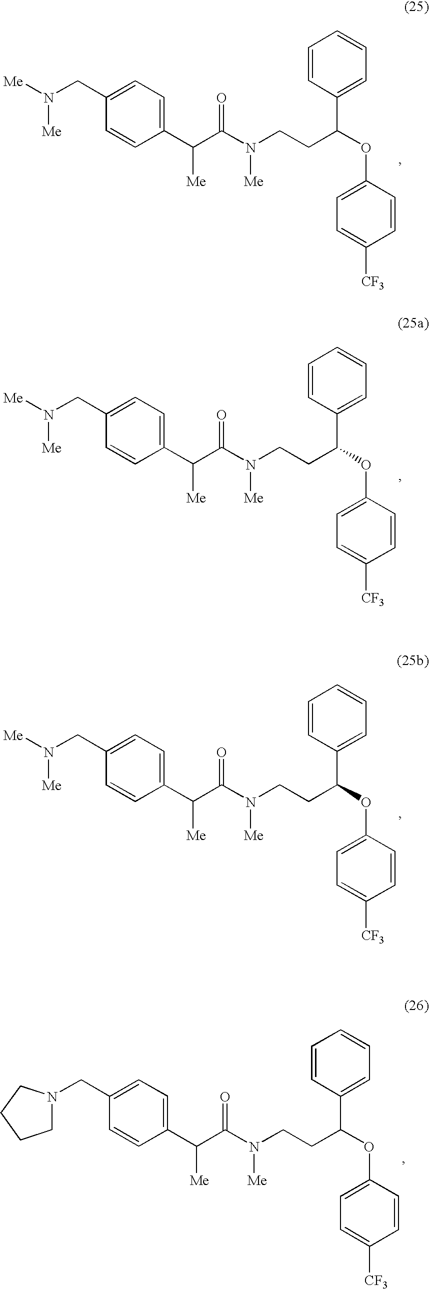

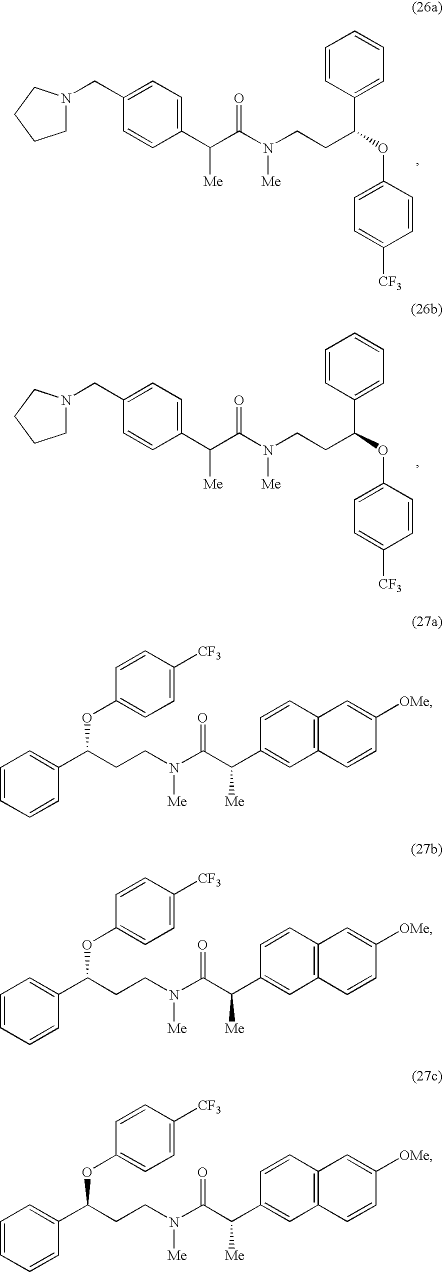

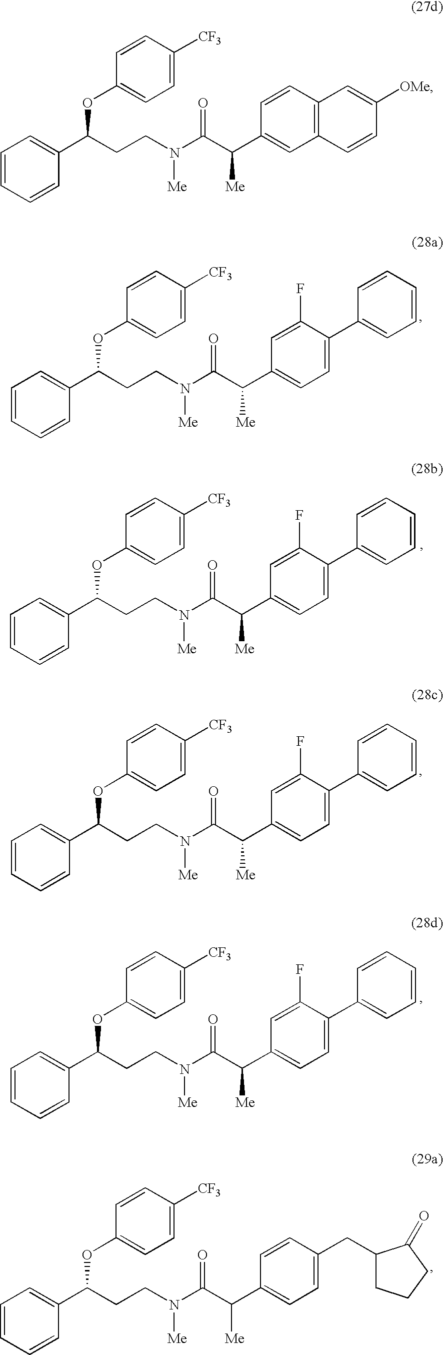

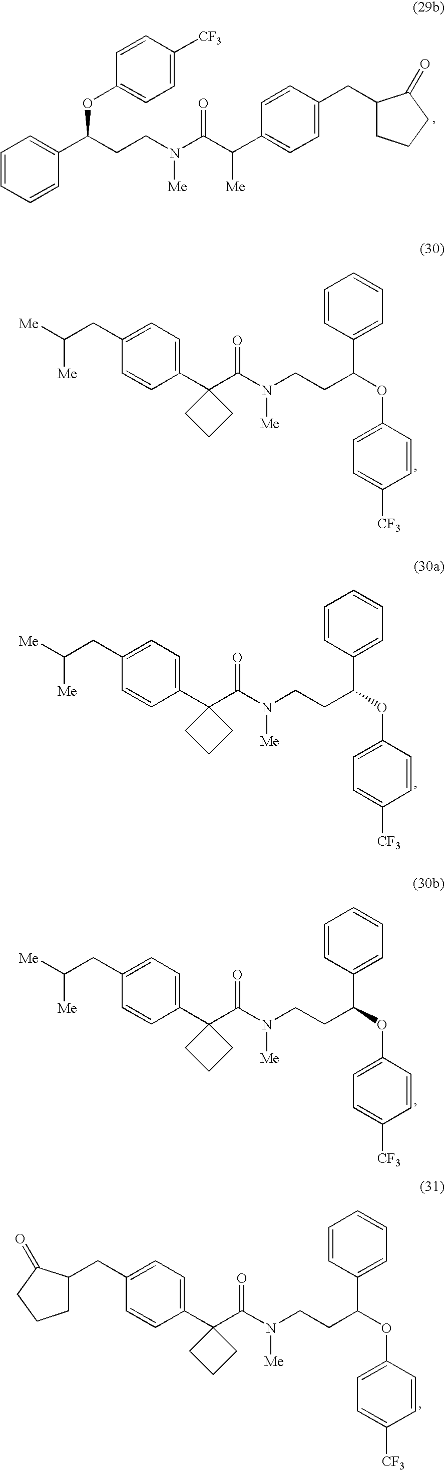

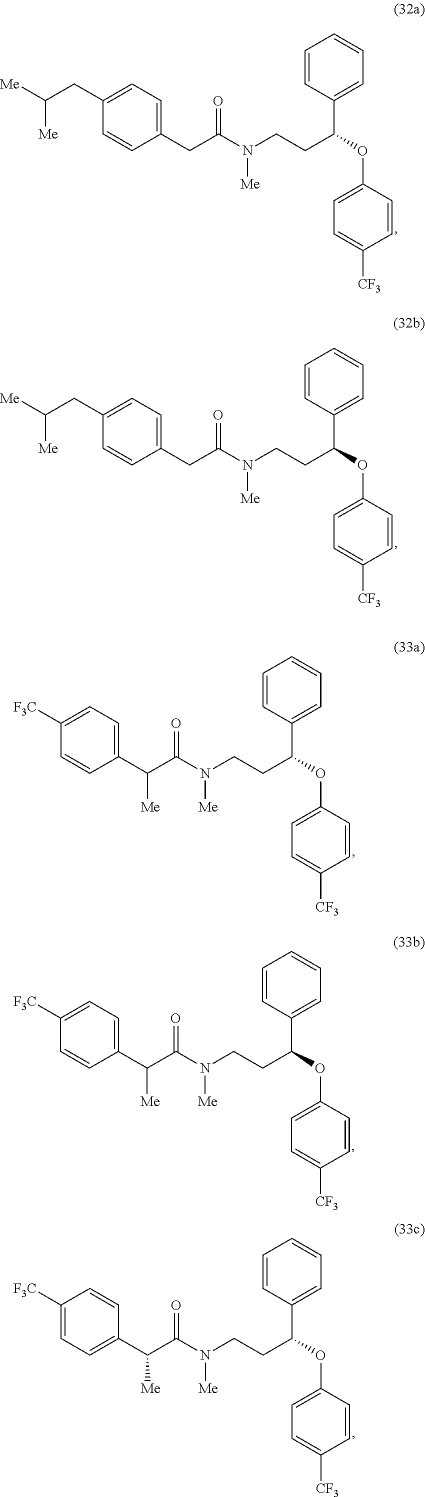

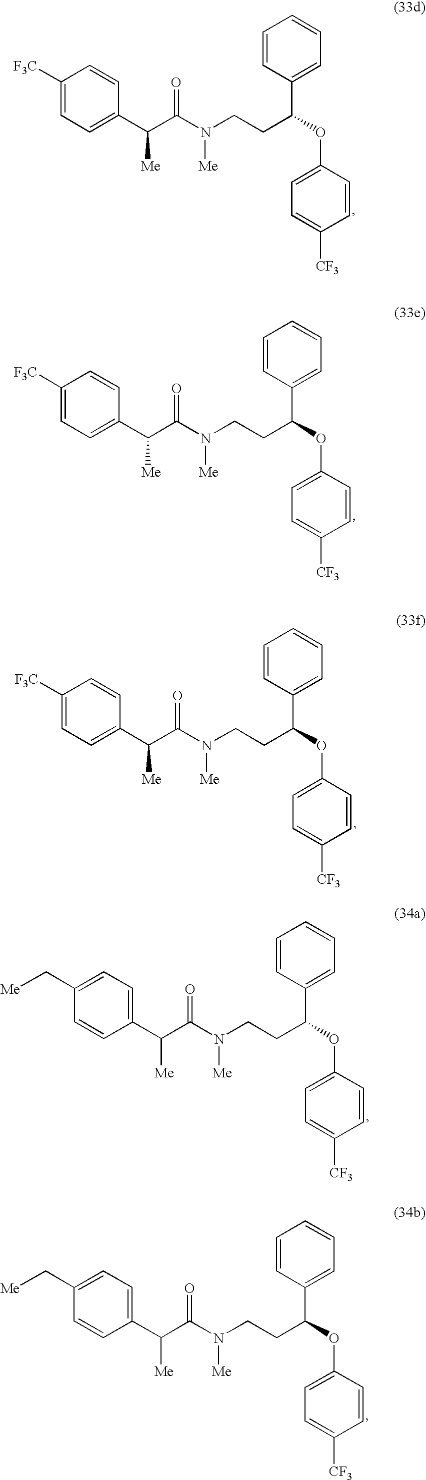

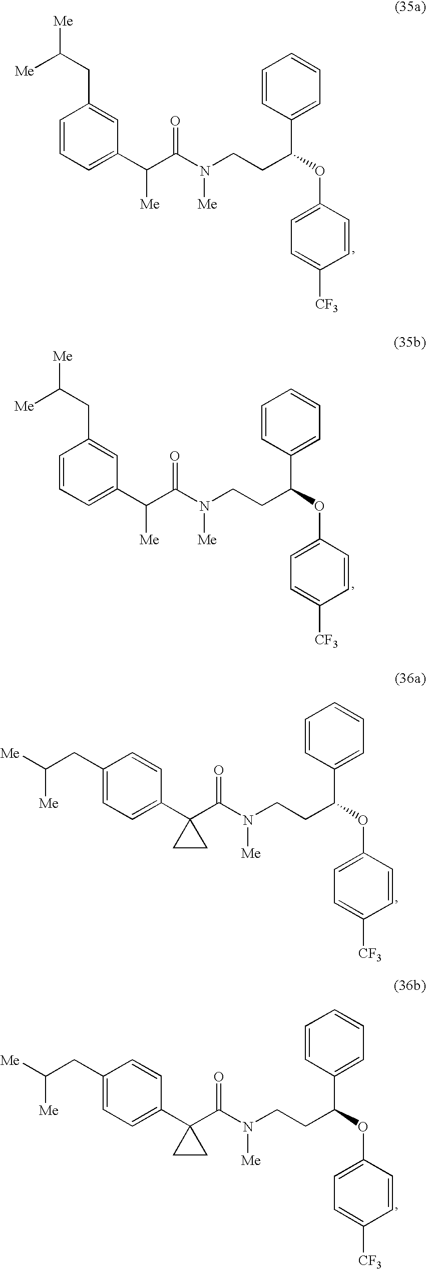

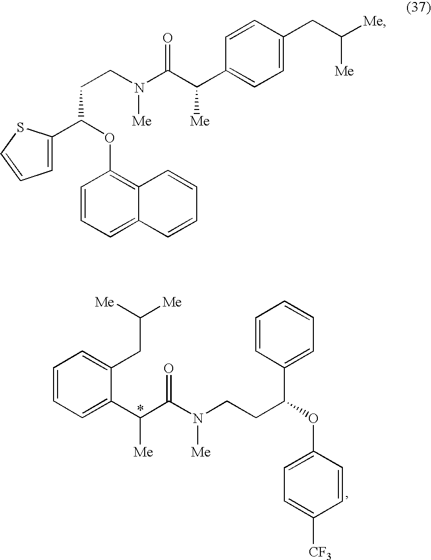

(mixture of diastereomers at indicated center, 38a and 38b), and

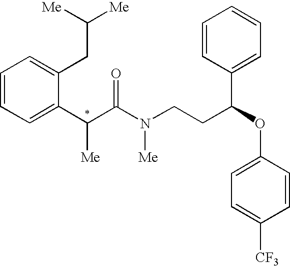

(mixture of diastereomers at indicated center, 38c and 38d).

The invention further provides compounds of formula III or a pharmaceutically acceptable salt thereof, or a solvate of the compound or its salt:

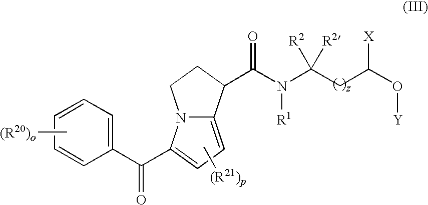

wherein:
X represents H or an optionally substituted aryl or heteroaryl ring system;
Y represents an optionally substituted aryl or heteroaryl ring system;
o represents an integer from 0 to 5;
p represents an integer from 0 to 2;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system; and
R²⁰ and R²¹ each independently for each occurrence represent a substituent.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R²⁰ and R²¹ each independently represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, o is 0.

In certain embodiments, p is 0.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, the compound of formula III is represented by formula IIIa or a pharmaceutically acceptable salt thereof, or a solvate of the compound or its salt:

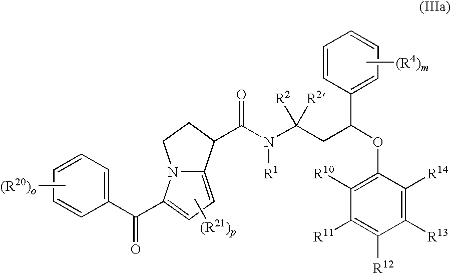

wherein:
m represents an integer from 0 to 5;
o represents an integer from 0 to 5;
p represents an integer from 0 to 2;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²⁰, and R²¹, each independently for each occurrence, represents a substituent; and
R¹⁰, R¹¹, R¹², R¹³, and R¹⁴ each independently represent hydrogen or a substituent.

In certain embodiments, R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R¹⁰, R¹¹, R¹³, and R¹⁴ are hydrogen, and R¹² is optionally substituted lower alkyl. In certain such embodiments, R¹² is trifluoromethyl.

In certain embodiments, R⁴, R²⁰, and R²¹ each independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, m is 0.

In certain embodiments, o is 0.

In certain embodiments, p is 0.

In certain embodiments, a compound of formula IIIa has the structure 5:

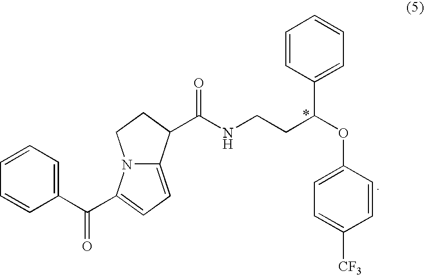

In certain embodiments, the compound of formula IIIa has the structure 5 and is enriched for the (R) configuration at the indicated position (*), e.g., compound 5a,

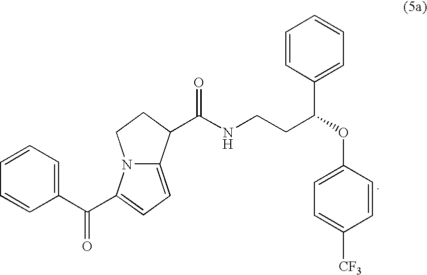

In certain such embodiments, the compound is substantially free of the (S) configuration at the indicated position (*).

Further exemplary compounds of formula IIIa include the following:

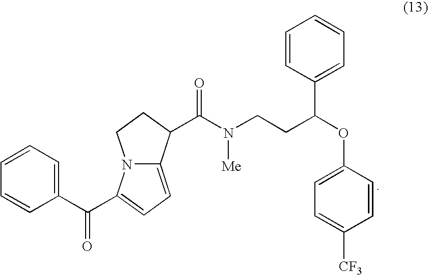

The invention further provides compounds of formula IV or a pharmaceutically acceptable salt thereof, or a solvate of the compound or its salt:

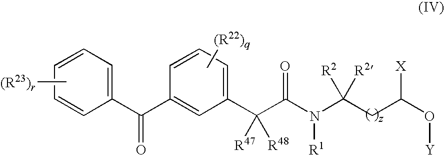

wherein:
X represents H or an optionally substituted aryl or heteroaryl ring system;
Y represents an optionally substituted aryl or heteroaryl ring system;
r represents an integer from 0 to 5;
q represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²² and R²³ each independently for each occurrence represent a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R²² and R²³ each independently represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, q is 0.

In certain embodiments, r is 0.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, R⁴⁷ is hydrogen and R⁴⁸ is an optionally substituted lower alkyl, such as methyl. In certain embodiments, R⁴⁷ and R⁴⁸ taken together with the carbon to which they are attached form a cyclopropane or cyclobutane ring.

In certain embodiments, the compound of formula IV is represented by formula IVa or a pharmaceutically acceptable salt thereof, or a solvate of the compound or its salt:

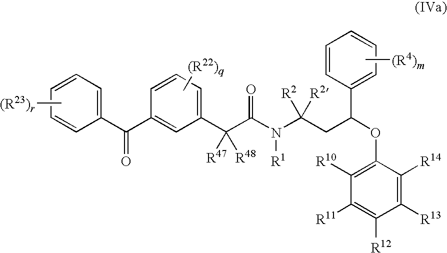

wherein:
m represents an integer from 0 to 5;
r represents an integer from 0 to 5;
q represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²², and R²³, each independently for each occurrence, represents a substituent;
R¹⁰, R¹¹, R¹², R¹³, and R¹⁴ each independently represent hydrogen or a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system.

In certain embodiments, R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R¹⁰, R¹¹, R¹³, and R¹⁴ are hydrogen, and R¹² is optionally substituted lower alkyl. In certain such embodiments, R¹² is trifluoromethyl.

In certain embodiments, R⁴, R²², and R²³ each independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R′ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, m is 0.

In certain embodiments, q is 0.

In certain embodiments, r is 0.

In certain embodiments, R⁴⁷ is hydrogen and R⁴⁸ is an optionally substituted lower alkyl, such as methyl. In certain embodiments, R⁴⁷ and R⁴⁸ taken together with the carbon to which they are attached form a cyclopropane or cyclobutane ring.

In certain embodiments, a compound of formula IVa has the structure 6:

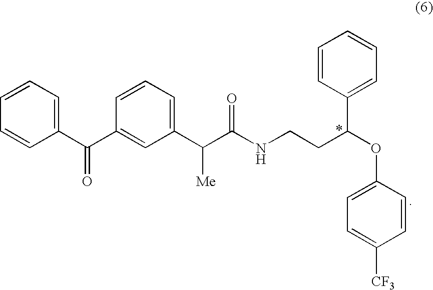

In certain embodiments, the compound of formula IVa has the structure 6 and is enriched for the (R) configuration at the indicated position (*). In certain such embodiments, the compound is substantially free of the (S) configuration at the indicated position (*).

Further exemplary compounds of formula IVa include the following:

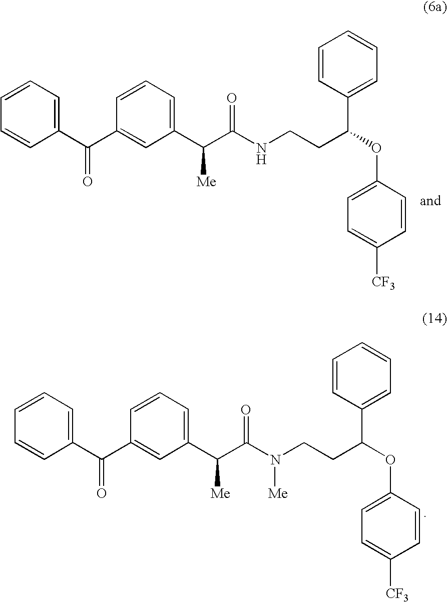

The invention further provides compounds of formula V or a pharmaceutically acceptable salt thereof, or a solvate of the compound or its salt:

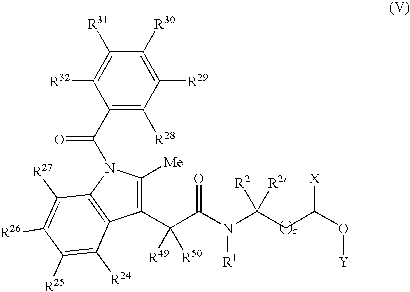

wherein:
X represents H or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R²⁴, R²⁵, R²⁶, R²⁷, R²⁸, R²⁹, R³⁰, R³¹, and R³² each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R²⁴, R²⁶, R²⁷, R²⁸, R²⁹, R³¹, and R³² each independently represent hydrogen, R²⁵ represents optionally substituted alkoxyl, and R³⁰ represents halogen. In certain such embodiments, R²⁵ represents methoxy, and R³⁰ represents chloro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, when z is 1, X represents an optionally substituted aryl or heteroaryl ring system. In certain embodiments, X represents an optionally substituted aryl or heteroaryl ring system.

In certain embodiments, R⁴⁹ and R⁵⁰ are each hydrogen. In certain embodiments, R⁴⁹ and R⁵⁰ taken together with the carbon to which they are attached form a cyclopropane or cyclobutane ring.

In certain embodiments, the compound of formula V is not one of the following:

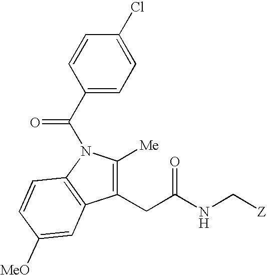

wherein Z is

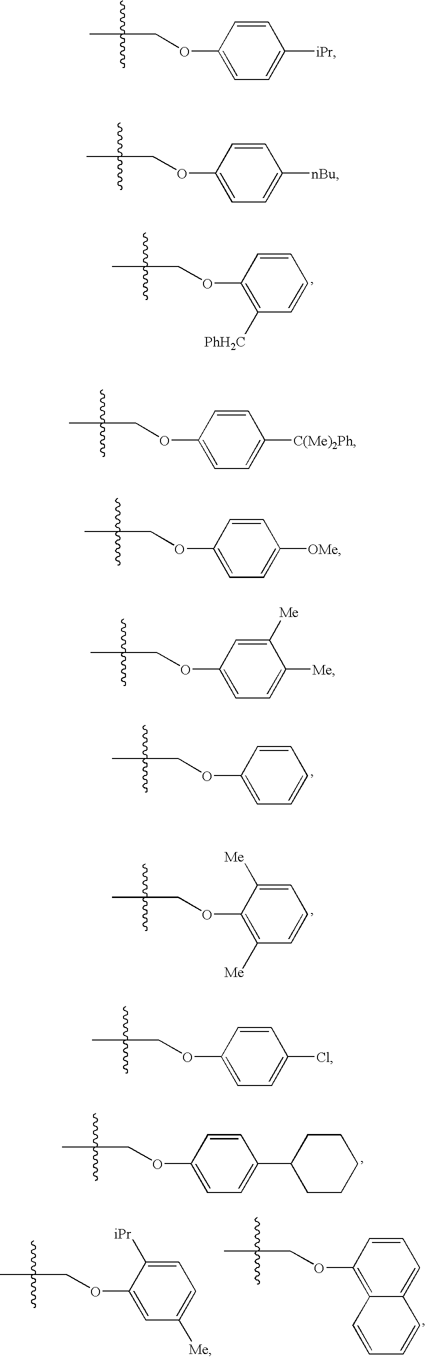

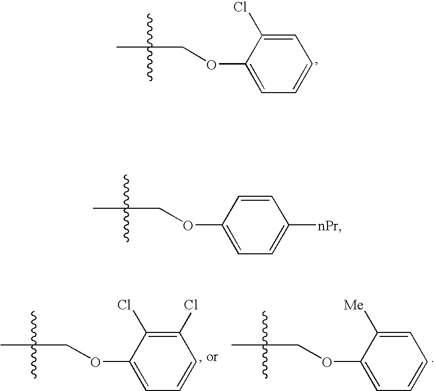

In certain embodiments, the compound of formula V is represented by formula Va or a pharmaceutically acceptable salt thereof, or a solvate of the compound or its salt:

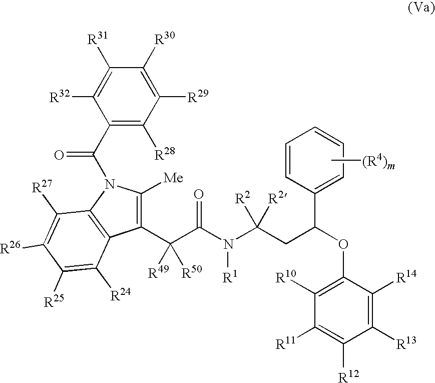

wherein:
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent;
R¹⁰, R¹¹, R¹², R¹³, R¹⁴, R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system.

In certain embodiments, R²⁴, R²⁵, R²⁶, R²⁷, R²⁸, R²⁹, R³⁰, R³¹, and R³² each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R²⁴, R²⁶, R²⁷, R²⁸, R²⁹, R³¹, and R³² each independently represent hydrogen, R²⁵ represents optionally substituted alkoxyl, and R³⁰ represents halogen. In certain such embodiments, R²⁵ represents methoxy, and R³⁰ represents chloro.

In certain embodiments R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R¹⁰, R¹¹, R¹³, and R¹⁴ are hydrogen, and R¹² is optionally substituted lower alkyl. In certain such embodiments, R¹² is trifluoromethyl.

In certain embodiments, R⁴, independently for each occurrence, represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, R¹ represents hydrogen.

In certain embodiments, R² and R^{2′} are each hydrogen. In certain embodiments, R² is hydrogen and R^{2′} is optionally substituted lower alkyl, such as methyl or —CH₂OCH₃.

In certain embodiments, m is 0.

In certain embodiments, R⁴⁹ and R⁵⁰ are each hydrogen. In certain embodiments, R⁴⁹ and R⁵⁰ taken together with the carbon to which they are attached form a cyclopropane or cyclobutane ring.

In certain embodiments, a compound of formula Va has the structure 7:

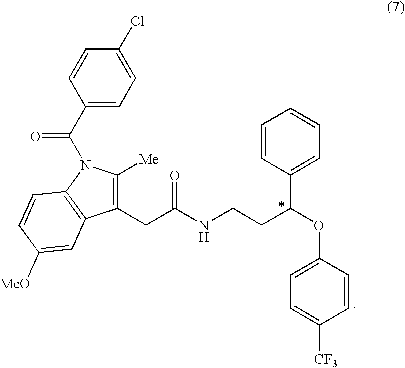

In certain embodiments, the compound of formula Va has the structure 7 and is enriched for the (R) configuration at the indicated position (*), e.g., compound 7a,

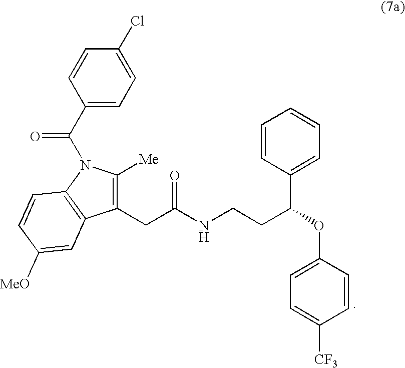

In certain such embodiments, the compound is substantially free of the (S) configuration at the indicated position (*).

Further exemplary compounds of formula Va include the following:

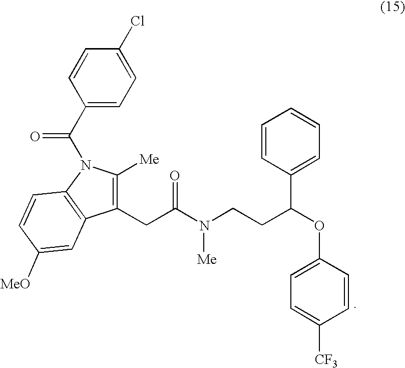

The invention further provides compounds of formula VI or a pharmaceutically acceptable salt thereof, or a solvate of the compound or its salt:

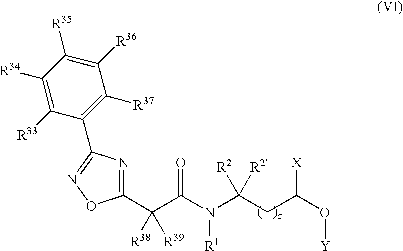

wherein:
X represents H, methyl, or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³³, R³⁴, R³⁵, R³⁶, and R³⁷ each independently represent hydrogen or a substituent; and
R³⁸ and R³⁹ each independently represent hydrogen or a substituent, or R³⁸ and R³⁹ taken together with the carbon to which they are attached form a three- to six-membered cyclic ring system.

In certain embodiments, X represents an optionally substituted aryl or heteroaryl ring system. In certain embodiments, X represents methyl or an optionally substituted aryl or heteroaryl ring system. In certain embodiments, X represents methyl or an optionally substituted aryl or heteroaryl ring system when Z is 1. In certain embodiments, X represents an optionally substituted aryl or heteroaryl ring system when Z is 1.

In certain embodiments wherein X is H, z is 0, 2, 3, or 4.

In certain embodiments, X represents an optionally substituted phenyl or thiophene. In certain such embodiments, X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.

In certain embodiments, Y represents an optionally substituted phenyl or napthyl ring system. In certain such embodiments, Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, Y is optionally substituted with optionally substituted lower alkyl. In certain such embodiments, Y is substituted with trifluoromethyl. In certain embodiments, Y is not substituted with aminoalkyl.

In certain embodiments, R³³, R³⁴, R³⁵, R³⁶, and R³⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R³³, R³⁴, R³⁵, R³⁶, and R³⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro, wherein at least one of R³³, R³⁴, R³⁵, R³⁶, and R³⁷ independently represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro. In certain embodiments, R³³, R³⁴, R³⁶, and R³⁷ are hydrogen, and R³⁵ is optionally substituted lower alkyl. In certain such embodiments, R³⁵ is trifluoromethyl or —CH₂CH(CH₃)₂.

In certain embodiments, R¹ represents hydrogen.

In certain embodiments, R² and R^{2′} are each hydrogen.

In certain embodiments, R³⁸ and R³⁹ each independently represent hydrogen, halo, optionally substituted lower alkyl, or optionally substituted piperidine. In certain embodiments, R³⁸ represents hydrogen and R³⁹ represents

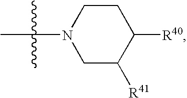

wherein:
R⁴⁰ represents hydrogen, optionally substituted lower alkyl, or optionally substituted amino, such as N-methyl-N-benzylamino; and
R⁴¹ represents hydrogen or optionally substituted lower alkyl, such as (4-(trifluoromethyl)phenoxy)methyl.

In certain embodiments, R³⁸ represents hydrogen and R³⁹ represents

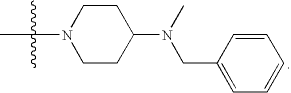

In certain embodiments, R³⁸ represents hydrogen and R³⁹ represents

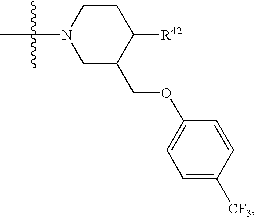

wherein R⁴² represents hydrogen or optionally substituted lower alkyl.

Exemplary compounds of formula VI include the following:

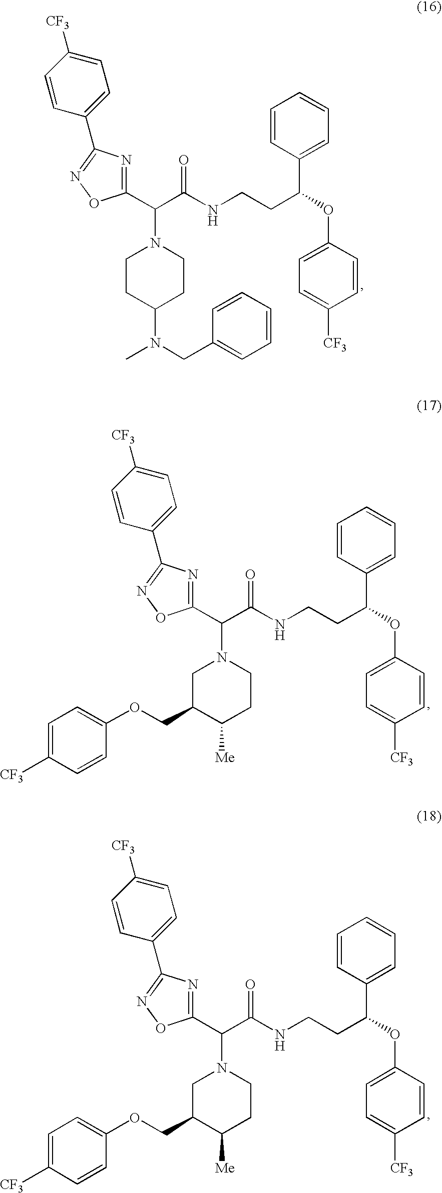

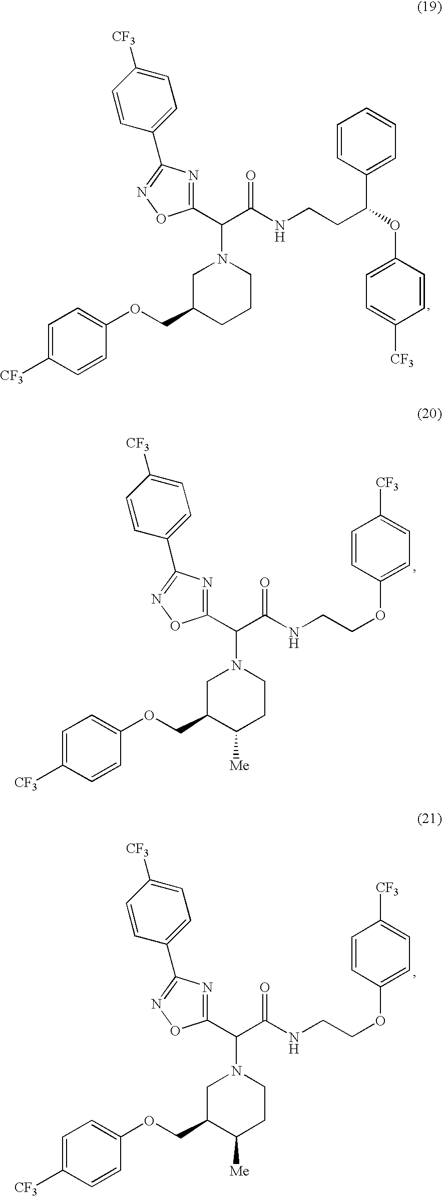

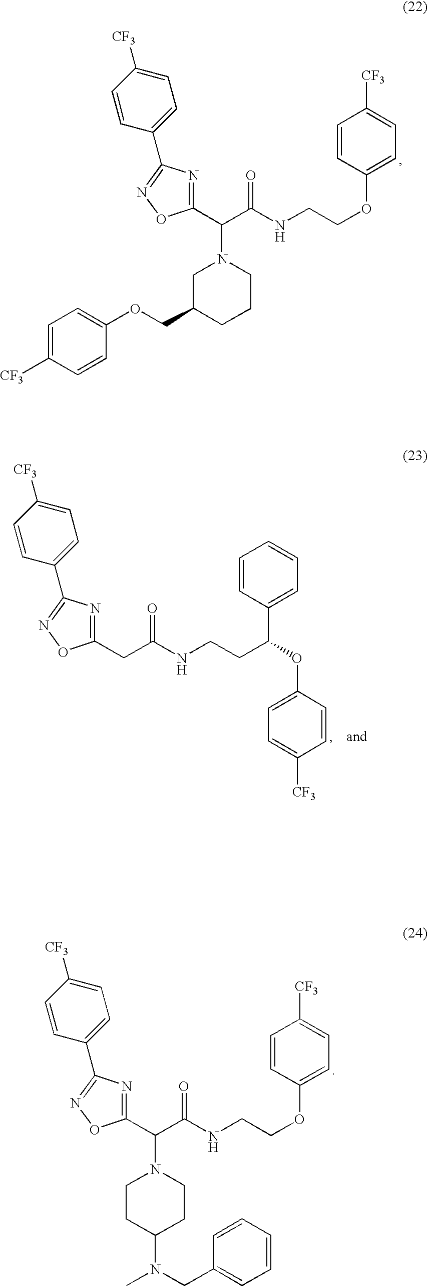

In certain embodiments, compounds of the invention may be racemic. In certain embodiments, compounds of the invention may be enriched in one enantiomer. For example, a compound of the invention may have greater than 30% ee, or 40% ee, or 50% ee, or 60% ee, or 70% ee, or 80% ee, or 90% ee, or even 95% or greater ee. In certain embodiments, compounds of the invention may be enriched in one or more diastereomer. For example, a compound of the invention may have greater than 30% de, or 40% de, or 50% de, or 60% de, or 70% de, or 80% de, or 90% de, or even 95% or greater de.

The present invention also relates to a method of treating obesity in a mammal. The invention further relates to a method of minimizing metabolic risk factors associated with obesity, such as hypertension, diabetes and dyslipidemia. In one embodiment, the methods comprise administering to a mammal in need of such treatment an effective anti-obesity dose of a compound of the invention (e.g., a compound of any of formulae I, Ia, II, IIa, III, IIIa, IV, IVa, V, Va, or VI or a pharmaceutically acceptable salt thereof, or a solvate of the compound or its salt).

In preferred embodiments of the methods of the invention, the mammal is a human.

In another aspect, the present invention provides a method of treating or preventing metabolic syndrome or a disorder associated with metabolic syndrome (e.g., obesity, diabetes, hypertension, and hyperlipidemia) in a mammal comprising administering to a mammal suffering from metabolic syndrome or a disorder associated with metabolic syndrome (e.g., obesity, diabetes, hypertension, and hyperlipidemia) an effective dose of a compound of the invention (e.g., a compound of any of formulae I, Ia, II, IIa, III, IIIa, IV, IVa, V, Va, or VI or a pharmaceutically acceptable salt thereof, or a solvate of the compound or its salt).

In certain embodiments, the disorder associated with metabolic syndrome is diabetes.

In preferred embodiments of the methods of the invention, the mammal is a human.

The present invention also relates to a method of treating depression in a mammal. In one embodiment, the methods comprise administering to a mammal in need of such treatment an effective anti-depressant dose of a compound of the invention (e.g., a compound of any of formulae I, Ia, II, IIa, III, IIIa, IV, IVa, V, Va, or VI or a pharmaceutically acceptable salt thereof, or a solvate of the compound or its salt).

In certain embodiments, the depression is endogenous depression, somatogenic depression, psychogenic depression, or depression in specific life situations. In certain such embodiments, the endogenous depression is unipolar depression (e.g., major depression or major depressive disorder) or bipolar depression. In certain embodiments, the somatogenic depression is organic depression, symptomatic depression, or pharmacogenic depression. In certain embodiments, the depression in specific life situations is postpartum depression, old-age depression, childhood depression, seasonal depression, or pubertal depression. In certain embodiments, the depression is treatment-refractory depression or resistant depression. In certain embodiments, the depression is dysthymia.

As used herein, the term “depression” includes major depressive disorder (including single episode and recurrent), unipolar depression, treatment-refractory depression, resistant depression, anxious depression and dysthymia (also referred to as dysthymic disorder). The term “depression” encompasses any major depressive disorder, dysthymic disorder, mood disorders due to medical conditions with depressive features, mood disorders due to medical conditions with major depressive-like episodes, substance-induced mood disorders with depressive features and depressive disorder not otherwise specific as defined by their diagnostic criteria, as listed in the Diagnostic and Statistical Manual of Mental Disorders, 4th Edition, Text Revision, American Psychiatric Association, 2000. Preferably, the depression is major depressive disorder, unipolar depression, treatment-refractory depression, resistant depression or anxious depression. More preferably, the depression is major depressive disorder.

In preferred embodiments of the methods of the invention, the mammal is a human.

In certain embodiments, the present invention relates to methods of treatment with a compound of any of formulae I, Ia, II, IIa, III, Ma, IV, IVa, V, Va, or VI or a pharmaceutically acceptable salt thereof, or a solvate of the compound or its salt. In certain embodiments, the therapeutic preparation may be enriched to provide predominantly one enantiomer of a compound (e.g., of formula I, Ia, V, Va, or VI). An enantiomerically enriched mixture may comprise, for example, at least 60 mol percent of one enantiomer, or more preferably at least 75, 90, 95, or even 99 mol percent. In certain embodiments, the compound of formula I or Ia has the structure 1. In certain such embodiments, the compound of formula I is enriched in the (R) enantiomer. In certain embodiments, the compound of formula 1 enriched in the (R) enantiomer is substantially free of the (S)-enantiomer, wherein substantially free means that the substance in question makes up less than 10%, or less than 5%, or less than 4%, or less than 3%, or less than 2%, or less than 1% as compared to the amount of the (R)-enantiomer, e.g., in the composition or compound mixture. For example, if a composition or compound mixture contains 98 grams of the (R)-enantiomer and 2 grams of the (S)-enantiomer, it would be said to contain 98 mol percent of the (R)-enantiomer and only 2% of the (S)-enantiomer. In certain embodiments, the compound of formula 1 is provided as a salt of the compound of formula 1 or a solvate of the compound of formula 1 or its salt.

In certain embodiments, the therapeutic preparation may be enriched to provide predominantly one diasteriomer of a compound (e.g., of formula II, IIa, III, IIIa, IV, or IVa). An diasteriomerically enriched mixture may comprise, for example, at least 60 mol percent of one diasteriomer, or more preferably at least 75, 90, 95, or even 99 mol percent.

Compounds suitable for use in methods of the invention include any compound of the invention as set forth above (e.g., a compound represented by any of formulae I, Ia, II, IIa, III, IIIa, IV, IVa, V, Va, or VI, or a pharmaceutically acceptable salt thereof, or a solvate of the compound or its salt).

One aspect of the present invention provides a pharmaceutical composition suitable for use in a human patient, or for veterinary use, comprising an effective amount of a compound of the invention (e.g., a compound of any of formulae I, Ia, II, IIa, III, IIIa, IV, IVa, V, Va, or VI, or a pharmaceutically acceptable salt thereof, or a solvate of the compound or its salt), and one or more pharmaceutically acceptable carriers. In certain embodiments, the pharmaceutical compositions may be for use in treating or preventing obesity, metabolic syndrome, a disorder associated with metabolic syndrome (e.g., obesity, diabetes, hypertension, and hyperlipidemia), or depression. In certain embodiments, the pharmaceutical preparations have a low enough pyrogen activity to be suitable for use in a human patient, or for veterinary use. In certain embodiments, the pharmaceutical preparation comprises an effective amount of a compound of the invention (e.g., a compound of any of formulae I, Ia, II, IIa, III, IIIa, IV, IVa, V, Va, or VI, or a pharmaceutically acceptable salt thereof, or a solvate of the compound or its salt).

Compounds of the invention (e.g., a compound of any of formulae I, Ia, II, IIa, III, IIIa, IV, IVa, V, Va, or VI, or a pharmaceutically acceptable salt thereof, or a solvate of the compound or its salt) may be used in the manufacture of medicaments for the treatment of any diseases disclosed herein.

As used herein, the term “obesity” includes both excess body weight and excess adipose tissue mass in an animal. An obese individual is one having a body mass index of ≧30 kg/m². While the animal is typically a human, the invention also encompasses the treatment of non-human mammals. The treatment of obesity, as provided in methods of the present invention, contemplates not only the treatment of individuals who are defined as “obese”, but also the treatment of individuals with weight gain that if left untreated may lead to the development of obesity.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “hydrate” as used herein, refers to a compound formed by the association of water with the parent compound.

The term “metabolite” is intended to encompass compounds that are produced by metabolism of the parent compound under normal physiological conditions. For example, an N-methyl group may be cleaved to produce the corresponding N-desmethyl metabolite, or an amide may be cleaved to the corresponding carboxylic acid and amine. Preferred metabolites of the present invention include those that exhibit activity suitable for the treatment of obesity, metabolic syndrome, or a disorder associated with metabolic syndrome.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “solvate” as used herein, refers to a compound formed by solvation (e.g., a compound formed by the combination of solvent molecules with molecules or ions of the solute).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover; the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

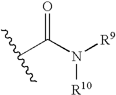

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

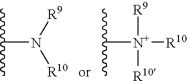

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

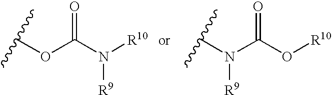

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

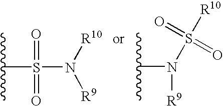

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R⁹, wherein R⁹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

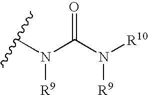

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

The amount of active agent(s) (e.g., a compound of the invention, such as a compound of any of formulae I, Ia, II, IIa, III, IIIa, IV, IVa, V, Va or VI) administered can vary with the patient, the route of administration and the result sought. Optimum dosing regimens for particular patients can be readily determined by one skilled in the art.

Compounds of the invention may be administered to an individual in need thereof. In certain embodiments, the individual is a mammal such as a human, or a non-human mammal. When administered to an individual, the compound of the invention can be administered as a pharmaceutical composition containing, for example, the compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free, or has low enough pyrogen activity. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

The term “low enough pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).

A pharmaceutically acceptable carrier can contain physiologically acceptable agents that act, for example, to stabilize or to increase the absorption of a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) containing a compound of the invention can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments a compound of the invention may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, a compound of the present invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In certain embodiments, a compound of the present invention (e.g., a compound of any of formulae I, Ia, II, IIa, III, IIIa, IV, IVa, V, Va, or VI or a pharmaceutically acceptable salt thereof, or a solvate of the compound or its salt) may be administered conjointly with another treatment for diabetes including, but not limited to, sulfonyl ureas (e.g., chlorpropamide, tolbutamide, glyburide, glipizide, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, or combinations of the above.

In certain embodiments, a compound of the present invention (e.g., a compound of any of formulae I, Ia, II, IIa, III, IIIa, IV, IVa, V, Va, or VI or a pharmaceutically acceptable salt thereof, or a solvate of the compound or its salt) may be administered conjointly with another treatment for obesity including, but not limited to, orlistat, sibutramine, phendimetrazine, phentermine, diethylpropion, benzphetamine, mazindol, dextroamphetamine, rimonabant, cetilistat, GT 389-255, APD356, pramlintide/AC137, PYY3-36, AC 162352/PYY3-36, oxyntomodulin, TM 30338, AOD 9604, oleoyl-estrone, bromocriptine, ephedrine, leptin, pseudoephedrine, or pharmaceutically acceptable salts thereof.

It is contemplated that a compound of the present invention will be administered to a subject (e.g., a mammal, preferably a human) in a therapeutically effective amount (dose). By “therapeutically effective amount” is meant the concentration of a compound that is sufficient to elicit the desired therapeutic effect (e.g., treatment of obesity, metabolic syndrome, or a disorder associated with metabolic syndrome, such as obesity, diabetes, hypertension, and hyperlipidemia). It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

As used herein, compounds of the invention (e.g., compounds of any of formulae I, Ia, II, IIa, III, IIIa, IV, IVa, V, Va, or VI, or a pharmaceutically acceptable salt thereof, or a solvate of the compound or its salt) include the pharmaceutically acceptable salts of compounds of the invention. The pharmaceutically acceptable salts of compounds of the invention can also exist as various solvates, such as with water, methanol, ethanol, dimethylformamide, and the like. Mixtures of such solvates can also be prepared. In general, the solvated forms are equivalent to unsolvated forms and are encompassed within the scope of the present invention. The source of such solvate can be from the solvent of crystallization, inherent in the solvent of preparation or crystallization, or adventitious to such solvent. Certain compounds of the present invention may exist in multiple crystalline or amorphous forms. In general, all physical forms are equivalent for the uses contemplated by the present invention and are intended to be within the scope of the present invention.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs form the various salt forms in certain physical properties, such as solubility in polar solvents, but otherwise the salts are equivalent to the parent form of the compound for the purposes of the present invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

Compounds of the invention (e.g., compounds of any of formulae I, Ia, II, IIa, III, IIIa, IV, IVa, V, Va, or VI, or a pharmaceutically acceptable salt thereof, or a solvate of the compound or its salt) may be synthesized using methods well-known in the art. For example, compounds of formula Ia may be synthesized by the formation of the amide from its corresponding amine (3) and carboxylic acid (2), as shown in Scheme 1. In certain embodiments, the carboxylic acid is converted to a carbonyl chloride before reacting with the amine.

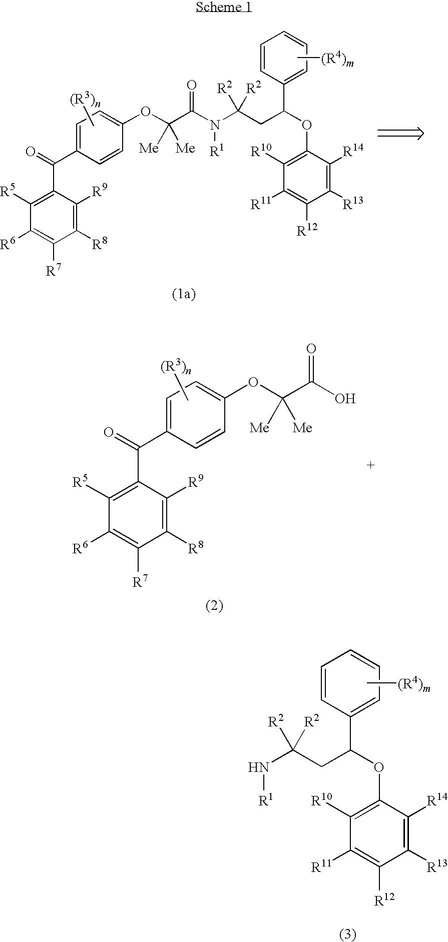

In certain embodiments, the amine (3) is norfluoxetine or an analog thereof. Norfluoxetine can be prepared by any of a number of methods generally known in the art. For example, there are several methods provided in the literature for making the racemate of norfluoxetine (U.S. Pat. No. 4,313,896). The racemate of norfluoxetine in turn can be resolved, if desired, into its (S) and (R) components by standard methods. In particular, norfluoxetine can be reacted with an enantiomerically pure chiral derivatizing agent, resolved on the basis of the different physicochemical properties of the diastereomeric derivatives, and then converted to the two separate enantiomers of norfluoxetine. One particularly preferred method of accomplishing this derivatization is analogous to that described in Robertson et al., J. Med. Chem., 31, 1412 (1988), wherein fluoxetine was reacted with an optically active form of 1-(1-naphthyl)ethyl isocyanate to form a urea derivative of fluoxetine. A similar mixture of norfluoxetine diastereomeric ureas can be separated through high pressure liquid chromatography into the individual diastereomers. Each individual diastereomer, in turn, can then be hydrolyzed to the individual enantiomers of norfluoxetine.

In certain embodiments, the carboxylic acid (2) is the carboxylic acid formed by hydrolysis of the ester of fenofibrate or an analog thereof. Fenofibrate can be prepared by any of a number of methods generally known in the art.

In certain embodiments, compounds of formula IIa may be synthesized by the formation of the amide from its corresponding amine and carboxylic acid. In certain embodiments, the amine is norfluoxetine or an analog thereof. In certain embodiments, the carboxylic acid is ibuprofen or an analog thereof. Ibuprofen can be prepared by any number of methods generally known in the art.

In certain embodiments, compounds of formula IIIa may be synthesized by the formation of the amide from its corresponding amine and carboxylic acid. In certain embodiments, the amine is norfluoxetine or an analog thereof. In certain embodiments, the carboxylic acid is ketorolac or an analog thereof. Ketorolac can be prepared by any number of methods generally known in the art.

In certain embodiments, compounds of formula IVa may be synthesized by the formation of the amide from its corresponding amine and carboxylic acid. In certain embodiments, the amine is norfluoxetine or an analog thereof. In certain embodiments, the carboxylic acid is ketoprofen or an analog thereof. Ketoprofen can be prepared by any number of methods generally known in the art.

In certain embodiments, compounds of formula Va may be synthesized by the formation of the amide from its corresponding amine and carboxylic acid. In certain embodiments, the amine is norfluoxetine or an analog thereof. In certain embodiments, the carboxylic acid is indomethacin or an analog thereof. Indomethacin can be prepared by any number of methods generally known in the art.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The present invention provides a kit comprising:
a) one or more single dosage forms each comprising a dose of a compound of any of formulae I, Ia, II, IIa, III, IIIa, IV, IVa, V, Va, or VI or a pharmaceutically acceptable salt thereof, or a solvate of the compound or its salt, and a pharmaceutically acceptable excipient; and
b) instructions for administering the single dosage forms for the treatment of obesity, metabolic syndrome, or a disorder associated with metabolic syndrome (e.g., obesity, diabetes, hypertension, and hyperlipidemia).

In certain embodiments, the invention relates to a method for conducting a pharmaceutical business, by manufacturing a formulation or kit as described herein, and marketing to healthcare providers the benefits of using the formulation or kit in the treatment of obesity, metabolic syndrome, or a disorder associated with metabolic syndrome (e.g., obesity, diabetes, hypertension, and hyperlipidemia).

In certain embodiments, the invention provides a method for conducting a pharmaceutical business, by providing a distribution network for selling a formulation or kit as described herein, and providing instruction material to patients or physicians for using the formulation to treat obesity, metabolic syndrome, or a disorder associated with metabolic syndrome (e.g., obesity, diabetes, hypertension, and hyperlipidemia).

In certain embodiments, the present invention relates to a method for conducting a pharmaceutical business, by providing a distribution network for selling a formulation or kit as described herein, and providing instruction material to patients or physicians for using the formulation to treat obesity, metabolic syndrome, or a disorder associated with metabolic syndrome (e.g., obesity, diabetes, hypertension, and hyperlipidemia).

In certain embodiments, the invention comprises a method for conducting a pharmaceutical business, by determining an appropriate formulation and dosage of a compound of the invention (e.g., a compound of any of formulae I, Ia, II, IIa, IIIa, IV, IVa, V, Va, or VI, or a pharmaceutically acceptable salt thereof, or a solvate of the compound or its salt) to be administered in the treatment of obesity, metabolic syndrome, or a disorder associated with metabolic syndrome (e.g., obesity, diabetes, hypertension, and hyperlipidemia), conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the invention (e.g., a compound of any of formulae I, Ia, II, IIa, III, IIIa, IV, IVa, V, Va, or VI, or a pharmaceutically acceptable salt thereof, or a solvate of the compound or its salt) to be administered in the treatment of obesity, metabolic syndrome, or a disorder associated with metabolic syndrome (e.g., obesity, diabetes, hypertension, and hyperlipidemia), and licensing, to a third party, the rights for further development and sale of the formulation.

The present invention provides a kit comprising:
a) one or more single dosage forms each comprising a dose of a compound of any of formulae I, Ia, II, IIa, III, IIIa, IV, IVa, V, Va, or VI, or a pharmaceutically acceptable salt thereof, or a solvate of the compound or its salt, and a pharmaceutically acceptable excipient; and
b) instructions for administering the single dosage forms for the treatment of depression.

In certain embodiments, the invention relates to a method for conducting a pharmaceutical business, by manufacturing a formulation or kit as described herein, and marketing to healthcare providers the benefits of using the formulation or kit in the treatment of depression.

In certain embodiments, the invention provides a method for conducting a pharmaceutical business, by providing a distribution network for selling a formulation or kit as described herein, and providing instruction material to patients or physicians for using the formulation to treat depression.

In certain embodiments, the present invention relates to a method for conducting a pharmaceutical business, by providing a distribution network for selling a formulation or kit as described herein, and providing instruction material to patients or physicians for using the formulation to treat depression.

In certain embodiments, the invention comprises a method for conducting a pharmaceutical business, by determining an appropriate formulation and dosage of a compound of the invention (e.g., a compound of any of formulae I, Ia, II, IIa, III, IIIa, IV, IVa, V, Va, or VI, or a pharmaceutically acceptable salt thereof, or a solvate of the compound or its salt) to be administered in the treatment of depression, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the invention (e.g., a compound of formula I or Ia, or a pharmaceutically acceptable salt thereof, or a solvate of the compound or its salt) to be administered in the treatment of depression, and licensing, to a third party, the rights for further development and sale of the formulation.

### EXEMPLIFICATION

### Example 1

### Radioligand Binding Assay for CB1 Activity

An assay to assess in vitro CB1 activity was performed according to reported procedures in Compton, D. R. et al., “Cannabinoid structure activity relationships: correlation of receptor binding and in vivo activities” J Pharmacol Exp Ther., 265(1): 218-226 and Rinaldi-Carmona M., et al., “Characterization of two cloned human CB1 cannabinoid receptor isoforms,” J Pharmacol Exp Ther. 278(2): 871-878 under the following conditions:

Source: Human recombinant HEK-293 cells

### Ligand: 0.5 nM [.H] CP-55,940

### Vehicle: 1% DMSO

Incubation Time/Temp: 90 minutes @ 37° C.

### Incubation Buffer: 50 mM Tris-HCl, pH 7.4, 1 mM EDTA, 3 mM MgCl., 0.5% BSA

### Non-Specific Ligand: 10 mM R(+)-WIN-55, 212-2

K_{D}: 1.3 nM (historical value)
Bmax: 0.7 pmole/mg Protein (historical value)
Specific Binding: 60% (historical value)

### Quantitation Method: Radioligand Binding

Significance Criteria: ≧50% of max stimulation or inhibition

A mixture of compounds 8a and 8b was tested in this assay and demonstrated an IC50 of 208 nM.

### Example 2

### Synthesis of Compounds of the Invention

Proton and carbon NMR spectra were obtained on a Bruker AC 300 spectrometer at 300 MHz and 75 MHz, respectively. Proton spectra were referenced to tetramethylsilane as an internal standard. Melting points were obtained on a Mel-Temp II apparatus and are uncorrected. HPLC analyses were obtained using an Alltech Alltima C18 Rocket Column Method A (Table 1) with UV detection using standard solvent programs on a Shimadzu Prominence HPLC system.

TABLE 1
HPLC (Method A):

| | Flow | | |
|---|---|---|---|
| Time | (mL/min) | % A | % B |
| 1.00 | 2.5 mL | 90 | 10 |
| 4.50 | 2.5 mL | 0 | 100 |
| 10.00 | 2.5 mL | 0 | 100 |
| 11.50 | 2.5 mL | 90 | 10 |

Alltech Altima C18 Rocket Column
A = Water with 0.05% v/v Trifluoroacetic Acid
B = Acetonitrile with 0.05% v/v Trifluoroacetic Acid
UV Detection at 254 nm

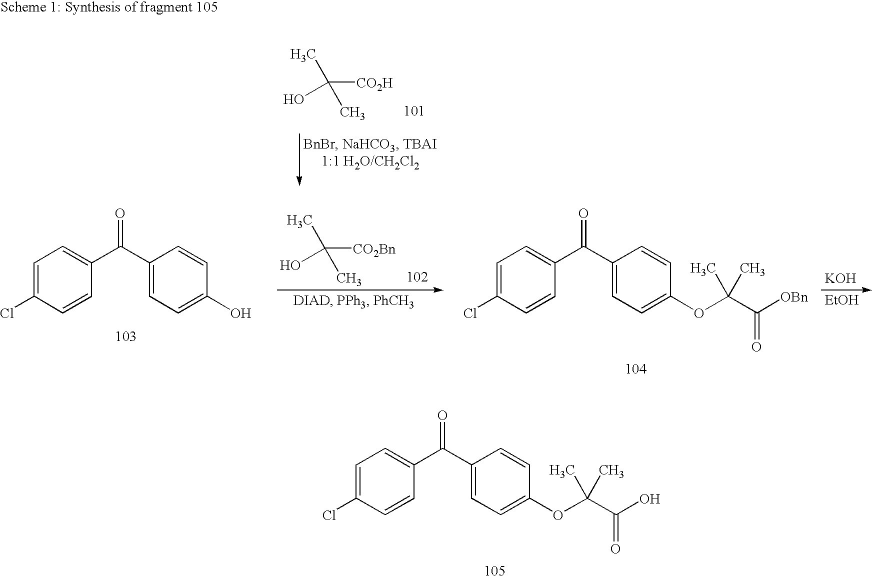

Preparation of Benzyl 2-Hydroxy-2-methylpropanoate (102): A solution of 2-hydroxy-2-methylpropanoic acid (101, 9.27 g, 89.1 mmol), sodium bicarbonate (7.49 g, 89.1 mmol), benzyl bromide (16.8 g, 98.0 mmol) and tetrabutylammonium iodide (32.9 g, 89.1 mmol) in H₂O/CH₂Cl₂ (1:1, 300 mL) was stirred at room temperature for 2.5 d. The reaction mixture was diluted with CH₂Cl₂ (200 mL) and the organic layer was separated. The aqueous layer was extracted with CH₂Cl₂ (2×50 mL) and the combined organic extracts were washed with brine, dried over Na₂SO₄, filtered, and concentrated in vacuo. Purification of the residue by flash column chromatography (silica gel, 7:3 hexanes/EtOAc) provided 102 (12.2 g, 70%) as a yellow oil: ¹H NMR (300 MHz, CDCl₃) δ 7.37-7.32 (m, 5H), 5.20 (s, 2H), 3.05 (s, 1H), 1.45 (s, 6H).

Preparation of Benzyl 2-[4-(4-Chlorobenzoyl)phenoxy]-2-methylpropanoate (104): A solution of 102 (8.77 g, 45.1 mmol) and diisopropyl azodicarboxylate (11.4 g, 56.4 mmol) in anhydrous toluene (120 mL) was added dropwise at 100° C. over 4 h to a mixture of (4-chlorophenyl)(4-hydroxyphenyl)methanone (103, 11.0 g, 47.4 mmol) and triphenylphosphine (14.8 g, 56.4 mmol) in anhydrous toluene (240 mL). The reaction mixture was stirred at 100° C. for 16 h. The reaction mixture was diluted with CH/Cl₂ (200 mL) and filtered through a plug of silica gel to remove polar side products. The remaining organic fractions were concentrated in vacuo. Purification of the residue by flash column chromatography (silica gel, 1-10% EtOAc in hexanes) provided 104 (8.14 g, 44%) as a pale yellow oil: ¹H NMR (300 MHz, CDCl₃) δ 7.69-7.62 (m, 4H), 7.44 (d, J=8.6 Hz, 2H), 7.31-7.25 (m, 5H), 6.77 (d, J=8.8 Hz, 2H), 5.20 (s, 2H), 1.67 (s, 6H).

Preparation of 2-[4-(4-Chlorobenzoyl)phenoxy]-2-methylpropanoic Acid (105): To a solution of 104 (5.00 g, 12.2 mmol) in ethanol (150 mL) was added a solution of potassium hydroxide (2.74 g, 48.9 mmol) in ethanol (50 mL) and the reaction mixture was heated at reflux and stirred for 16 h. Ethanol was removed under vacuum and water (200 mL) was added. The aqueous solution was washed with diethyl ether (2×100 mL) and acidified to pH 1 with 2 N HCl. The acidic aqueous solution was then extracted with ethyl acetate (2×150 mL) and the combined organic extracts were washed with brine, dried over Na₂SO₄, filtered, and concentrated in vacuo to provide 105 (3.57 g, 91%) as an off-white solid: ¹H NMR (300 MHz, CD₃OD) δ 7.75-7.70 (m, 4H), 7.52 (d, J=8.6 Hz, 2H), 6.96 (d, J=9.0 Hz, 2H), 1.65 (s, 6H).

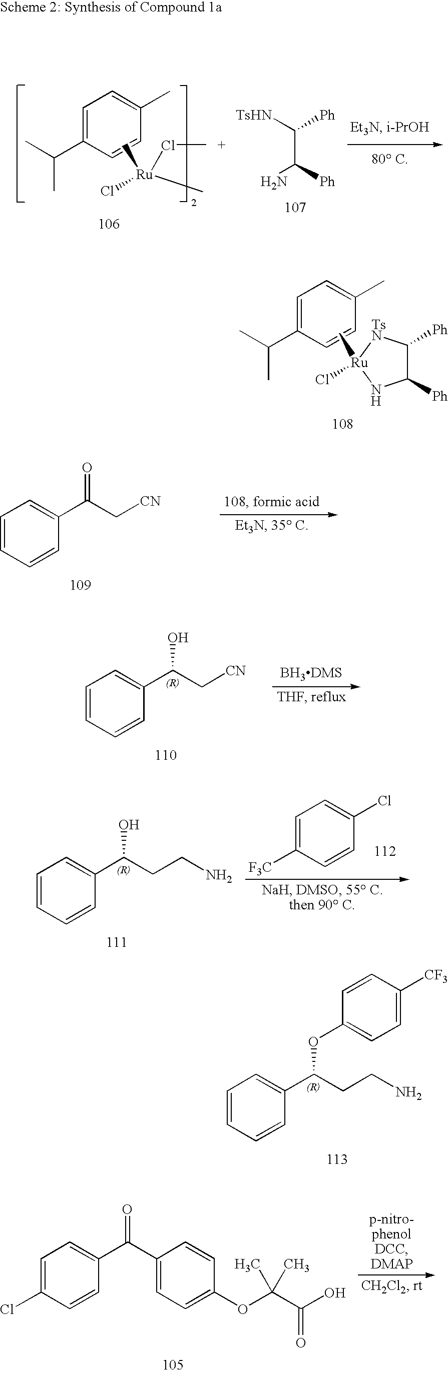

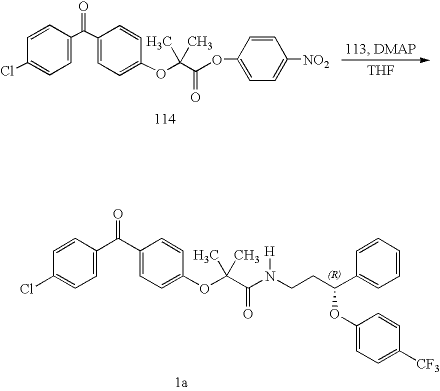

Preparation of (R)-RuCl [(1R,2R)-p-TsNCH(C₆H₅)CH(C₆H₅)NH₂](η6-cymene) (108): A mixture of [RuCl₂(η6-cymene)]₂ (106, 400 mg, 0.65 mmol), (1R,2R)-N-p-toluenesulfonyl-1,2-diphenylethylenediamine (107, 500 mg, 1.36 mmol), and triethylamine (0.40 mL, 2.80 mmol) in 2-propanol (10 mL) was heated at 80° C. for 1 h. The orange solution was concentrated in vacuo and the solid ruthenium complex was washed with a small amount of water. The filter cake was dried under reduced pressure to afford 108 (820 mg, 91%) as a brown solid: ¹H NMR (300 MHz, CDCl₃) δ 7.16-7.03 (m, 5H), 6.79-6.51 (m, 9H), 5.69-5.64 (m, 4H), 3.88-3.80 (m, 1H), 3.67-3.58 (m, 1H), 3.19-3.01 (m, 1H), 2.38 (s, 3H), 2.23 (s, 3H), 1.39 (s, 6H).

Preparation of (R)-3-Hydroxy-3-phenylpropanenitrile (110): A mixture of triethylamine (1.25 mL, 8.90 mmol) and formic acid (0.40 mL, 12.4 mmol) was added to 2-cyanoacetophenone (109, 498 mg, 3.40 mmol) and ruthenium catalyst 108 (5.0 mg, 0.008 mmol). The mixture was then stirred at 30-35° C. for 2.5 d, after which, the reaction mixture was neutralized with saturated NaHCO₃ (2 mL) and diluted with ethyl acetate (5 mL). The organic layer was washed with water (4 mL), dried over Na₂SO₄, filtered, and concentrated in vacuo. The residue was purified by flash column chromatography (silica gel, 3:1 hexanes/EtOAc) to afford 110 (377 mg, 76%) as an amorphous solid: ¹H NMR (300 MHz, CDCl₃) δ 7.40-7.35 (m, 5H), 5.09-5.03 (m, 1H), 2.79-2.77 (m, 2H), 2.35 (d, J=14.4 Hz, 1H); [α]²³_{D} +53.1°: (c 0.82, ethanol).

Preparation of (R)-3-Amino-1-phenylpropan-1-ol (111): A solution of borane-dimethyl sulfide complex (2.0 M in THF, 4.5 mL, 9.0 mmol) was added dropwise to a solution of 110 (1.2 g, 8.16 mmol) in anhydrous THF (5 mL) at 0° C. under nitrogen. The mixture was heated at 70° C. for 4 h. After cooling to 0° C., 15 mL of methanol was carefully added and the reaction mixture was evaporated to a residue, which was taken up in 2 N NaOH (20 mL) and extracted with 2-propanol (3×15 mL). The combined organic layers were dried over Na₂SO₄, filtered, and concentrated in vacuo. The crude residue was purified by flash column chromatography (silica gel, 10:1:0.01 EtOAc/MeOH/concentrated ammonium hydroxide) to afford 111 (920 mg, 76%) as an amorphous solid: ¹H NMR (300 MHz, CDCl₃) δ 7.32-7.21 (m, 5H), 4.99-4.94 (m, 1H), 3.48-3.46 (m, 1H), 3.10-3.06 (m, 1H), 3.00-2.92 (m, 1H), 2.47 (br s, 2H), 1.87-1.72 (m, 2H).

Preparation of (R)-3-Phenyl-3-[4-(trifluoromethyl)phenoxy]propan-1-amine (113): To a solution of 111 (0.78 g, 5.16 mmol) in anhydrous DMSO (25 mL) at 0° C. was added sodium hydride (0.32 g, 8.0 mmol, 60% in mineral oil) and was then heated at 55° C. for 30 min to form the alkoxide. To the alkoxide was slowly added 4-chlorobenzotrifluoride (112, 1.4 g, 7.6 mmol) and the resultant mixture was heated at 90° C. for 2 h. After cooling to room temperature, the mixture was diluted with 2 N NaOH (40 mL) and was extracted with diethyl ether (3×20 mL). The combined organic layers were dried over Na₂SO₄, filtered, and concentrated in vacuo. The crude residue was purified by flash column chromatography (silica gel, 100:1: EtOAc/MeOH/Et₃N) to afford 113 (1.18 g, 78%) as an amorphous solid: ¹H NMR (300 MHz, CDCl₃) δ 7.43 (d, J=14.0 Hz, 2H), 7.35-7.24 (m, 5H), 6.90 (d, J=14.0 Hz, 2H), 5.32 (dd, J=14.0, 8.0 Hz, 1H), 2.89 (t, J=11.5 Hz, 1H), 2.26-2.14 (m, 1H), 2.05-1.93 (m, 1H).

Preparation of 4-Nitrophenyl 2-[4-(4-Chlorobenzoyl)phenoxy]-2-methylpropanoate (114): To a solution of 105 (980 mg, 3.1 mmol) and p-nitrophenol (535 mg, 3.83 mmol) in CH₂Cl₂ (80 mL) was added N,N-dicyclohexylcarbodiimide (833 mg, 4.04 mmol) and 4-dimethylaminopyridine (92 mg, 0.75 mmol), and the mixture was stirred at room temperature for 2 h. The reaction mixture was directly passed through a plug of silica gel eluting with 8:1 hexanes/EtOAc. The crude white solid 114 (1.50 g) was used directly in the next coupling step without further purification.

Preparation of (R)-2-[4-(4-Chlorobenzoyl)phenoxy]-2-methyl-N-[3-phenyl-3-(4-trifluoromethyl)phenoxy]propyl)propanamide (1a): To a solution of 113 (456 mg, 1.55 mmol) and 114 (816 mg, 1.69 mmol) in THF (30 mL) was added 4-dimethylaminopyridine (66 mg, 0.54 mmol) and the mixture was stirred at room temperature for 2 h. The reaction mixture was quenched with 2 N NaOH (40 mL) and was extracted with ethyl acetate (3×20 mL). The combined organic layers were dried over Na₂SO₄, filtered, and concentrated in vacuo. The residue was purified by flash column chromatography (silica gel, 6:1 hexanes/EtOAc) to afford 1a (480 mg, 63%) as an amorphous solid: ¹H NMR (300 MHz, CDCl₃) δ 7.76-7.66 (m, 4H), 7.45-7.36 (m, 4H), 7.31-7.19 (m, 5H), 6.97-6.78 (m, 5H), 5.19 (t, J=10.5 Hz, 1H), 3.60-3.42 (m, 2H), 2.20-2.12 (m, 2H), 1.60 (s, 6H); ¹³C NMR (75 MHz, CDCl₃) δ 173.6, 159.6, 158.3, 139.7, 138.4, 135.8, 131.6, 131.3, 130.9, 128.7, 128.4, 128.1, 127.9, 126.6, 126.5, 125.7, 125.3, 119.1, 115.4, 81.7, 78.9, 37.6, 36.4, 25.0; ESI MS m/z 618 [M+Na]⁺.

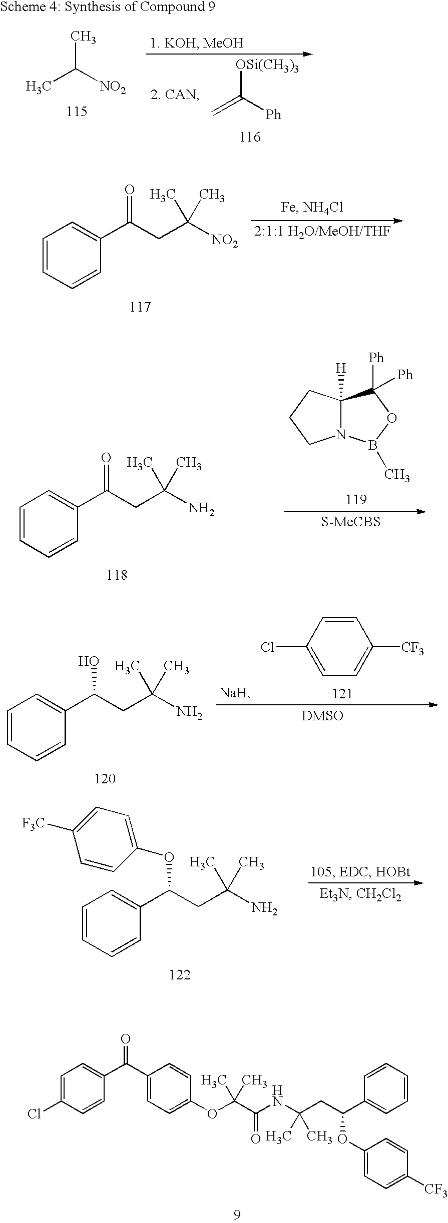

Preparation of 3-Methyl-3-nitro-1-phenylbutan-1-one (117): To a solution of cerium(IV) ammonium nitrate (11.7 g, 21.3 mmol) in anhydrous methanol (55 mL) was added a solution of trimethyl(1-phenylvinyloxy)silane (116, 1.83 g, 9.54 mmol) in anhydrous methanol (90 mL) at −78° C. under a nitrogen atmosphere. At the same temperature, a mixture of potassium hydroxide (0.819 g, 14.6 mmol) and 2-nitropropane (115, 1.00 g, 11.2 mmol) in MeOH (90 mL) was gradually added. After 15 min, the reaction was quenched by adding 0.1 M Na₂S₂O₃ (10 mL) and water (100 mL). The mixture was extracted with CH₂Cl₂ (4×100 mL) and the combined extracts were dried over Na₂SO₄, filtered, and concentrated in vacuo to provide 117 (1.40 g, crude) as a yellow solid: ¹H NMR (300 MHz, CD₃OD) δ 7.99-7.96 (m, 2H), 7.64-7.59 (m, 1H), 7.52-7.46 (m, 2H), 3.81 (s, 2H), 1.72 (s, 6H); ESI m/z 239 [M+MeOH]⁺.

Preparation of 3-Amino-3-methyl-1-phenylbutan-1-one (118): To a solution of 117 (500 mg, 2.41 mmol) in H₂O/MeOH/THF (2:1:1, 24 mL) was added iron powder (674 mg, 12.1 mmol) and ammonium chloride (258 mg, 4.82 mmol). The reaction mixture was stirred overnight at room temperature and then heated to 55°C. for an additional 3 h. The reaction mixture was quenched with saturated NaHCO₃ (50 mL) and filtered through a plug of diatomaceous earth and the plug was washed with EtOAc (50 mL). The organic extract was dried over Na₂SO₄, filtered, and concentrated in vacuo. The residue was taken up in Et₂O (20 mL) and 2 N HCl (20 mL) was added. The resultant solution was stirred and Et₂O was evaporated under vacuum. The resultant mixture was extracted with ethyl acetate (2×20 mL). The acidic aqueous layer was basified to pH 10 with 1 M NaOH and the resultant mixture was extracted with ethyl acetate (3×20 mL). The combined organic extracts were dried over Na₂SO₄ and concentrated under vacuum to provide 118 (100 mg, 14% over 2 steps) as a yellow oil: ¹H NMR (300 MHz, CDCl₃) δ 7.95-7.92 (m, 2H), 7.58-7.53 (m, 1H), 7.48-7.42 (m, 2H), 3.07 (s, 2H), 1.27 (s, 6H).

Preparation of (R)-3-Amino-3-methyl-1-phenylbutan-1-ol (120): To a solution of (S)-Me-CBS (119, 1 M in toluene, 0.10 mL, 0.10 mmol) and borane-tetrahydrofuran complex (1.0 M in THF, 1.3 mL, 1.3 mmol) in anhydrous THF (1 mL) at 0° C. was added dropwise 118 (68 mg, 0.38 mmol) in anhydrous THF (3 mL) under a nitrogen atmosphere. The mixture was warmed to room temperature and stirred for 4 h. The reaction mixture was poured onto 2 N NaOH (5 mL) and the mixture was extracted with ethyl acetate (2×8 mL) and diethyl ether (2×8 mL). The combined organic layers were dried over Na₂SO₄, filtered, and concentrated in vacuo. The residue was purified by flash column chromatography (silica gel, 10:1:0.01 EtOAc/MeOH/concentrated ammonium hydroxide) to afford 120 (40 mg, 59%) as an amorphous solid: ¹H NMR (300 MHz, CDCl₃) δ 7.38-7.22 (m, 5H), 5.01 (dd, J=18.0, 4.5 Hz, 1H), 1.74-1.56 (m, 2H), 1.36 (s, 6H), 1.22 (s, 6H).

Preparation of (R)-2-Methyl-4-phenyl-4-[4-(trifluoromethyl)phenoxy]butan-2-amine (122): To a solution of 120 (59 mg, 0.33 mmol) in anhydrous DMSO (3 mL) at room temperature was added sodium hydride (60% in mineral oil, 50 mg, 1.25 mmol) and the reaction mixture was heated at 55° C. for 1 h to form the alkoxide. To the alkoxide was slowly added 4-chlorobenzotrifluoride (121, 76 mg, 0.43 mmol) and the resultant mixture was heated at 90° C. for 2 h. After cooling to room temperature, the mixture was diluted with 1 N NaOH (10 mL) and extracted with diethyl ether (3×10 mL). The combined organic layers were dried over Na₂SO₄, filtered, and concentrated in vacuo. The residue was purified by flash column chromatography (silica gel, 10:1:0.1 EtOAc/MeOH/concentrated ammonium hydroxide) to afford 122 (52 mg, 49%) as an amorphous solid: ¹H NMR (300 MHz, CDCl₃) δ 7.43 (d, J=14.5 Hz, 2H), 7.34-7.24 (m, 5H), 6.90 (d, J=14.5 Hz, 2H), 5.43 (dd, J=16.5, 4.0 Hz, 1H), 2.26-2.17 (m, 1H), 1.79 (dd, J=25.0, 4.0 Hz, 1H), 1.24 (s, 6H), 1.21 (s, 6H).

Preparation of (R)-2-[4-(4-Chlorobenzoyl)phenoxy]-2-methyl-N-{2-methyl-4-phenyl-4-[4-(trifluoromethyl)phenoxy]butan-2-yl}propanamide (9): To a suspension of 105 (78 mg, 0.24 mmol) in CH₂Cl₂ (5 mL) was added EDC.HCl (46 mg, 0.24 mmol), HOBT (36 mg, 0.27 mmol), 122 (52 mg, 0.16 mmol), and triethylamine (25 mg, 0.25 mmol). The reaction mixture was stirred at room temperature for 4 h. The reaction mixture was diluted with CH₂Cl₂ (5 mL), washed with water (5 mL) and br sine (10 mL), dried over Na₂SO₄, filtered, and concentrated in vacuo. Purification of the residue by flash column chromatography (silica gel, 7:1 hexanes/EtOAc) provided 9 (75 mg, 75%) as an amorphous solid: ¹H NMR (300 MHz, CDCl₃) δ 7.71-7.66 (m, 4H), 7.47-7.42 (m, 2H), 7.37-7.32 (m, 2H), 7.31-7.22 (m, 5H), 7.13 (s, 1H), 6.89-6.79 (m, 4H), 5.31 (dd, J=9.6, 1.8 Hz, 1H), 2.27 (dd, J=15.3, 9.8 Hz, 1H), 2.15 (dd, J=15.2, 2.0 Hz, 1H), 1.60 (s, 3H), 1.52 (s, 3H), 1.51 (s, 3H), 1.48 (s, 3H); ¹³C NMR (75 MHz, CDCl₃) δ 194.1, 173.4, 159.4, 159.4, 158.6, 141.0, 136.1, 131.8, 131.4, 131.2, 129.0, 128.6, 128.0, 126.9, 126.8, 126.8, 126.7, 126.0, 125.4, 123.5, 123.0, 119.3, 115.8, 82.1, 77.7, 53.3, 49.9, 27.9, 25.9, 25.8, 24.7; ESI m/z 646 [M+Na]⁺.

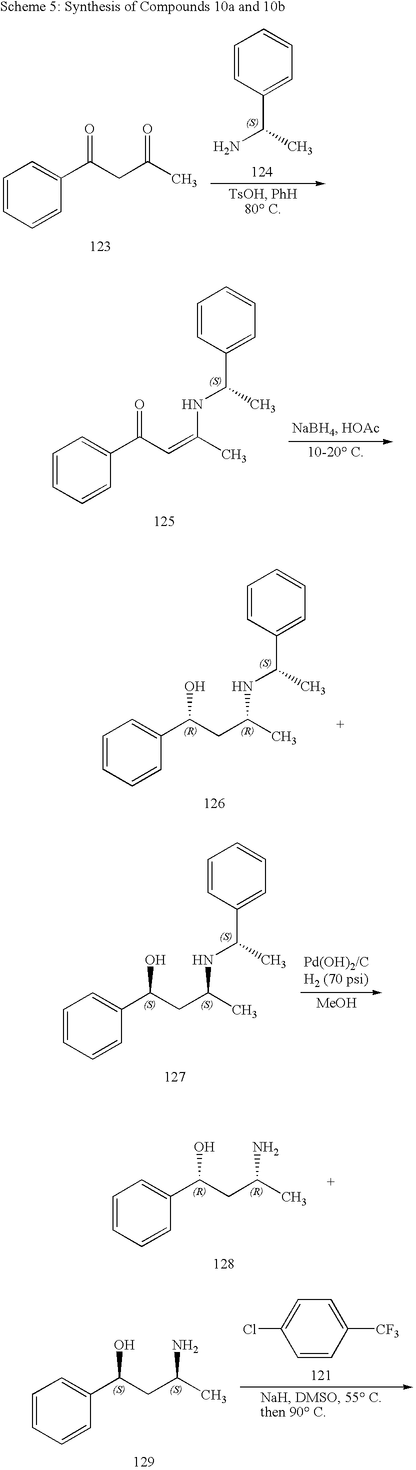

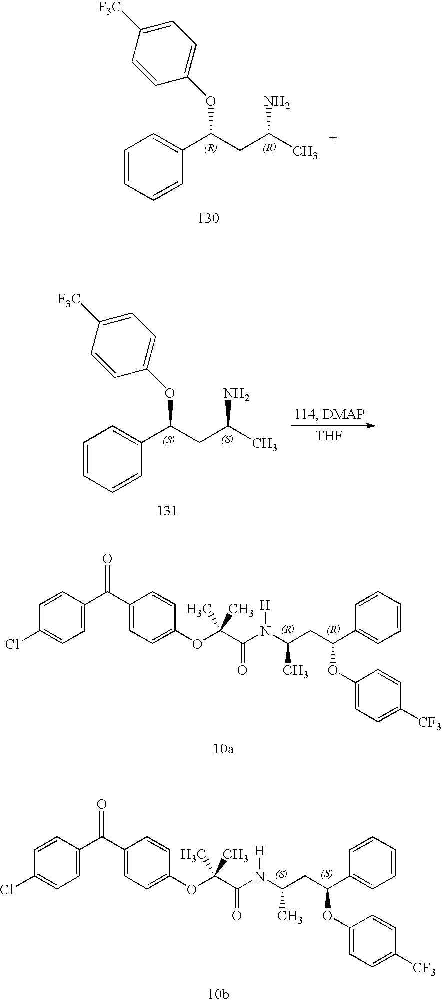

Preparation of (S,Z)-1-Phenyl-3-(1-phenylethylamino)but-2-en-1-one (125): To a solution of 1-phenyl-1,3,-butanedione (123, 4.14 g, 25.6 mmol) and (5)-α-methylbenzylamine (124, 3.56 g, 29.4 mmol) in benzene (80 mL) at room temperature was added a catalytic amount of p-toluenesulfonic acid monohydrate (110 mg), and the reaction mixture was heated at 115° C. for 4 d with azeotropic removal of water. The reaction was quenched with 1 N NaOH (40 mL) and extracted with diethyl ether (3×20 mL). The combined organic layers were dried over Na₂SO₄, filtered, and concentrated in vacuo. The residue was purified by flash column chromatography (silica gel, 10:1 hexanes/EtOAc) to afford 125 (6.78 g, 99%) as a colorless oil: ¹H NMR (300 MHz, CDCl₃) δ 7.90-7.87 (m, 2H), 7.37-7.21 (m, 8H), 5.70 (s, 1H), 4.77-4.72 (m, 1H), 1.94 (s, 3H), 1.61 (d, J=11.5 Hz, 3H).

Preparation of (1R,3R)-1-Phenyl-3-(S)-1-(phenylethylamino)butan-1-ol (126/127): To a solution of 125 (6.66 g, 25 mmol) in acetic acid (60 mL) was slowly added sodium borohydride (3.6 g, 90 mmol) at 0° C. The reaction was stirred at 0-20° C. for 3 h. The reaction mixture was made basic (pH 7) by adding aqueous NaOH (30%) to adjust to pH>7 and then extracted with CH₂Cl₂ (3×20 mL). The combined organic layers were dried over Na₂SO₄, filtered, and concentrated in vacuo. The residue was purified by flash column chromatography (silica gel, 10:1 hexanes/2-propanol) to afford 126/127 (3.74 g, 56%) as a 7:2 diastereomeric mixture: ¹H NMR (300 MHz, CDCl₃) δ 7.68-7.61 (m, 2H), 7.37-7.19 (m, 8H), 4.90 (dd, J=10.3, 2.6 Hz, 1H), 4.24-4.16 (m, 2H), 3.32-3.23 (m, 1H), 1.85-1.74 (m, 1H), 1.56-1.46 (m, 3H), 1.12 (d, J=11.5 Hz, 3H).

Preparation of (1R,3R)-3-Amino-1-phenylbutan-1-ol (128)/(1S,3S)-3-amino-1-phenylbutan-1-ol (129): A mixture of 126/127 (1.47 g, 3.72 mmol, 7:2 ratio of diastereomers) and 20% palladium(II) hydroxide on carbon (1.05 g) in methanol (20 mL) was subjected to hydrogenation conditions (70 psi) at room temperature for 48 h. The catalyst was filtered off through a plug of diatomaceous earth and the plug was washed with 1:2 MeOH/CHCl₃. The solvent was evaporated to give a crude yellow oil which was purified by flash column chromatography (silica gel, 100:1:0.01 EtOAc/MeOH/concentrated ammonium hydroxide) to give 128/129 (46 mg, 93%) as a 7:2 diastereomeric mixture: ¹H NMR (300 MHz, CDCl₃) δ 7.34-7.21 (m, 5H), 4.90 (dd, J=10.3, 3.7 Hz, 1H), 3.22-3.15 (m, 2H), 3.03 (br s, 2H), 1.78-1.71 (m, 1H), 1.54-1.42 (m, 2H), 1.16 (d, J=10:5 Hz, 3H).

Preparation of (2R,4R)-4-Phenyl-4-[4-trifluoromethyl)phenoxy]butan-2-amine (130)/(2S,4S)-4-Phenyl-4-[4-trifluoromethyl)phenoxy]butan-2-amine (131): To a solution of 128/129 (0.54 g, 3.27 mmol, 7:2 ratio of diastereomers) in anhydrous DMSO (20 mL) at 0° C. was added sodium hydride (60% in mineral oil, 0.22 g, 5.55 mmol) and the reaction mixture was heated at 55° C. for 30 min to form the alkoxide. To the alkoxide was slowly added 4-chlorobenzotrifluoride (121, 1.02 g, 5.52 mmol) and the resultant mixture was heated at 90° C. for 2 h. After cooling to room temperature, the mixture was diluted with 1 N NaOH (20 mL) and extracted with diethyl ether (3×15 mL). The combined organic extracts were dried over Na₂SO₄, filtered, and concentrated in vacuo. The residue was passed through a plug of silica gel eluting with 100:1:1 EtOAc/MeOH/Et₃N. Crude 130/131 (568 mg, 56%, 7:2 ratio of diastereomers) was isolated as an oily solid and was used directly in next coupling step without further purification.

Preparation of 2-[4-(4-Chlorobenzoyl)phenoxy]-2-methyl-N-[(2R,4R) and (2R,4S)-4-phenyl-4-(4-trifluoromethyl)phenoxy]butan-2-yl)propanamide (10a/10b): To a solution of 130/131 (568 mg, 1.84 mmol, 7:2 ratio of diastereomers) and 114 (680 mg (90%), 1.39 mmol) in THF (30 mL) was added 4-dimethylaminopyridine (95 mg, 0.78 mmol) and the mixture was stirred at room temperature overnight. The reaction was quenched with 2 N NaOH (30 mL) and extracted with ethyl acetate (3×20 mL). The combined organic layers were dried over Na₂SO₄, filtered, and concentrated in vacuo. The residue was purified by flash column chromatography (silica gel, 6:1 hexanes/EtOAc) to afford 10a/10b (589 mg, 63%, 7:2 ratio of diastereomers) as an amorphous solid: ¹H NMR (300 MHz, CDCl₃) δ 7.74-7.66 (m, 4H), 7.45-7.36 (m, 4H), 7.31-7.22 (m, 5H), 6.95-6.92 (m, 2H), 6.82 (d, J=8.6 Hz, 2H), 6.53 (d, J=8.2 Hz, 1H), 5.17 (dd, J=8.0, 4.9 Hz, 1H), 4.32-4.21 (m, 1H), 2.28-2.23 (m, 1H), 1.98-1.86 (m, 1H), 1.59 (s, 3H), 1.53 (s, 3H), 1.18 (d, J=5.6 Hz, 3H); ¹³C NMR (75 MHz, CDCl₃) δ 171.5, 158.0, 156.5, 138.6, 136.8, 134.1, 129.9, 129.8, 129.3, 127.1, 126.7, 126.2, 124.8, 123.9, 120.9, 117.7, 113.8, 80.0, 76.1, 43.6, 23.7, 22.8, 18.9; ESI MS m/z 632 [M+Na]⁺.

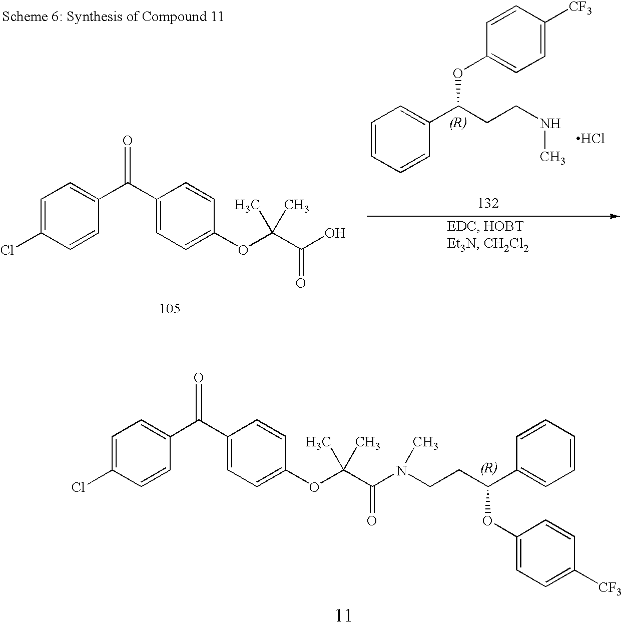

Preparation of (R)-[4-(4-Chlorobenzoyl)phenoxy]-N,2-dimethyl-N-[3-phenyl-3-(4-trifluoromethyl)phenoxy]propanamide (11): To a suspension of 105 (126 mg, 0.397 mmol) in CH₂Cl₂ (20 mL) was added EDC.HCl (83 mg, 0.433 mmol), HOBT (59 mg, 0.433 mmol), (R)-fluoxetine (132, 125 mg, 0.361 mmol), and triethylamine (44 mg, 0.433 mmol). The reaction mixture was stirred at room temperature for 16 h. The reaction mixture was diluted with CH₂Cl₂ (20 mL), washed with water (20 mL) and br sine (20 mL), dried over Na₂SO₄, filtered, and concentrated in vacuo. Purification of the residue by flash column chromatography (silica gel, 7:3 hexanes/EtOAc) provided 11 (160 mg, 66%) as a white amorphous solid: ¹H NMR (300 MHz, CDCl₃) δ 7.72-7.65 (m, 4H), 7.44-7.38 (m, 4H), 7.34-7.25 (m, 4H), 6.91-6.81 (m, 4H), 5.15-5.11 (m, 1H), 3.54-3.48 (m, 2H), 3.08-2.91 (m, 3H), 2.34-2.09 (m. 2H), 1.68-1.65 (m, 6H); ¹³C NMR (75 MHz, CDCl₃) δ 170.3, 157.4, 138.4, 136.5, 134.3, 130.3, 129.2, 128.4, 127.09, 126.9, 126.6, 126.1, 124.9, 124.8, 123.7, 123.5, 114.4, 113.7, 79.5, 76.4, 45.6, 34.0, 33.6, 23.8, 23.7; ESI m/z 632 [M+MeOH]⁺.

### Example 3

### Pharmacokinetic Analysis of Compounds of the Invention

The pharmacokinetics of compounds 4a, 6a, and 12 was evaluated in male Sprague-Dawley rats (n=3) following single intravenous (i.v., 1 mg/kg) or oral (p.o., 20 mg/kg) doses. Table 2 summarizes the dosing parameters and resulting pharmacokinetic measurements.

TABLE 2

| | Compound 4a | Compound 6a | Compound 12 |
|---|---|---|---|
| Mean Dosed | 0.497 | 0.496 | 0.489 |
| Volume (i.v., mL) | | | |
| t_{1/2} (i.v., hr) | 0.6 | 1.4 | 1.7 |
| Mean Dosed | 1.267 | 1.25 | 1.25 |
| Volume (p.o., mL) | | | |
| t_{1/2} (p.o., hr) | 1.0 | 2.1 | 1.4 |
| Oral Bioavailability | 5.1 | 14.8 | 6.5 |

### Example 4

### Binding Assays for Compounds of the Invention

A FLIPR assay was conducted to monitor agonist and antagonist selectivity for compounds of the invention against the CBI and GLP-1 receptors. Percentage activation and percentage inhibition values were determined for each compound on each of the GPCRs listed above. Agonist selectivity was determined upon initial addition of compounds followed by 10 minute incubation at 25° C. Following compound incubation, reference agonists were added at EC80 to determine percentage inhibition.

### Assay Design:

Agonist percentage activation determinations were obtained by assaying sample compounds and referencing the Emax control for each of the GPCRs profiled. Antagonist percentage inhibition determinations were obtained by assaying sample compounds and referencing the control EC80 wells for each of the GPCRs profiled. The protocol design is as follows:

Unless specified otherwise, all sample compounds were diluted in 100% anhydrous DMSO including all serial dilutions. Occasionally sample compounds were provided in a different solvent, in this case all master stock dilutions were performed in the specified diluent. All control wells contained identical solvent final concentrations as sample compound wells.

Sample compounds were transferred from a master stock solution into a daughter plate that was used in the assay. Each sample compound was diluted into assay buffer (1×HBSS with 20 mM HEPES) at an appropriate concentration to obtain final concentrations.

### Calcium Flux Assay

### Agonist Assay Format:

All sample compounds were plated at 1 μM in duplicate for assaying against all GPCRs. Reference agonists were handled as mentioned above serving as assay control. These reference agonists were handled as described above for both EMax and EC80 control wells. Assay was read for 90 seconds using the FLIPRTETRA. (This assay run added sample compounds and reference agonist to respective rows.) At the completion of the first “Single Addition” assay run, assay plate was removed from the FLIPRTetra and placed at 25° C. for 10 minutes.

### Antagonist Assay Format:

Using the EC50 values determined previously, stimulated all pre-incubated sample compound and reference antagonist (if applicable) wells with EC80 of reference agonist. Read for 90 seconds using the FLIPRTETRA. (This assay added reference agonist to respective wells—then fluorescence measurements were collected to calculate percentage inhibition.)

### Data Processing:

All plates were subjected to appropriate baseline corrections. Once baseline corrections were processed, maximum fluorescence values were exported and data manipulated to calculate percentage activation, percentage inhibition, Z′, EC50, and IC50. Percentage activation data was calculated using Emax as 100% control. Percentage inhibition was calculated using EC80 as 0% inhibition.

### Ligands Used:

### GPCR Target Reference Ligand

### CB1 CP-55,940

### GLP-1 Glucagon

### QC Criteria:

The QC criterium for percent effect validation was that duplicates must be <30% divergent. If this QC condition failed, that concentration was removed from curve fitting. If two or more concentrations with the dose response failed, the entire compound curve for that compound was repeated on a different assay plate.

The QC criterium for Z′ Statistic was that it must be >0.5. If this QC condition failed, all data collected from that particular GPCR was repeated.

The QC criterium for Signal-to-Noise (S/N) was that it must be >5. If this QC condition failed, all data collected from that particular GPCR was repeated.

The QC criterium for R2 was that it must be >0.90. If this QC condition failed, all data collected from that particular GPCR was repeated.

The QC criterium for EC50 Value for reference agonist(s) was that it must be within 5-fold of historic EC50 value. If this QC condition failed, all data collected from that particular GPCR was repeated.

The QC criterium for IC₅₀ Value for reference antagonist(s) was that it must be within 10-fold of historic EC50 value. If this QCcondition failed, all data collected from that particular GPCR was repeated.

Table 3 shows the results of these binding assays for several compounds of the invention.

TABLE 3

| | | | % Inhibition | % Inhibition |
|---|---|---|---|---|
| | % Activation | % Activation | Antagonist | Antagonist |
| Compound | Agonist CB1 | Agonist GLP-1 | CB1 | GLP-1 |
| 16 | 6.9 | 21.3 | | |
| 17 | 8.6 | 9.4 | | |
| 19 | 0.5 | 6.5 | | |
| 23 | 7.8 | 11.9 | | |
| 18 | 6.2 | 20.6 | | |
| 20 | 1.9 | 7.5 | 14.5 | −14.4 |
| 4a | 3.2 | 39.9 | 7.7 | −21.8 |
| 7a | −0.5 | 4.2 | 46.9 | 0 |
| 12 | 4.1 | 44.6 | 3.2 | −14.9 |
| 14 | 1.2 | −2.4 | 56.6 | 17.2 |
| 15 | 0 | −0.2 | 34.4 | −5.1 |
| 5a | 0.3 | 0.4 | 48.7 | −6.3 |
| 13 | 0.9 | 0.8 | 37.7 | −15.4 |
| 6a | 4.2 | 38.6 | 77.9 | −7.2 |
| 24 | 1.6 | 5.9 | 4.6 | −1.6 |
| 12a | 3.6 | 7.6 | 52.2 | 4.5 |
| 12b | 8.3 | 24.5 | 1.6 | 5.9 |
| 27a | −1.8 | −0.2 | 15.1 | 10.6 |
| 27b | −1.4 | 1.2 | 53.0 | 11.8 |
| 27c | 0.4 | 0.9 | 19.0 | 8.9 |
| 27d | 12.2 | 21.8 | 36.2 | 0.9 |
| 28a | 0.2 | 3.4 | 27.3 | 17.0 |
| 28b | −1.5 | 1.5 | 68.8 | −3.8 |
| 28c | −1.6 | 5.7 | 17.8 | 14.0 |
| 28d | 2.6 | 9.8 | 34.7 | 10.0 |
| 29a | −1.0 | 6.7 | 98.4 | −4.5 |
| 29b | −0.2 | 6.2 | 31.2 | 0.0 |
| 25a | 0.8 | −2.7 | 79.9 | 5.7 |
| 25b | 0.2 | −3.3 | 15.2 | 6.9 |
| 26a | −1.7 | −3.5 | 75.6 | 4.7 |
| 26b | −2.0 | −3.5 | 37.1 | −1.7 |
| 30a | 1.0 | −3.7 | 84.6 | 7.5 |
| 30b | −0.8 | −3.4 | 48.3 | 9.7 |
| 32a | 4.5 | 2.9 | 90.7 | 0.8 |
| 32b | 2.1 | −1.8 | 23.0 | −0.8 |
| 33a | −0.3 | 0.9 | 90.7 | 11.4 |
| 33b | 0.5 | 17.6 | 45.8 | ~0 |
| 33c | −0.9 | 0.8 | 80.1 | 0.2 |
| 33d | 1.4 | 0.6 | 82.8 | 11.4 |
| 33e | 1.7 | 0.4 | 0.8 | 9.4 |
| 33f | −0.5 | −3.5 | 34.0 | 11.5 |
| 34a | −2.9 | −0.8 | 99.0 | −1.0 |
| 34b | −1.9 | 0.2 | 76.4 | 1.6 |
| 35a | −1.0 | −0.9 | 80.5 | 7.7 |
| 35b | 1.1 | 3.8 | 38.9 | 0.3 |
| 36a | −1.2 | 4.4 | 59.9 | 11.0 |
| 36b | −1.1 | 0.5 | 15.5 | 5.5 |
| 37 | −0.9 | −0.3 | −16.3 | 2.0 |
| 38a | | | 98.5 | −20.2 |

### Example 5

### In Vivo Analysis of Compounds of the Invention in the db/db Mouse

Compounds 6a, 12a, and 12b were administered by oral gavage (100 and 200 mg/kg) once daily for three consecutive days to groups of 6 non-insulin dependent diabetic mellitus (NIDDM) male mice (C57BL/KsJ-db/db Jcl) weighing 40±2 gm (8-10 weeks old; serum glucose=500±50 mg/dl, serum insulin=7.0±0.5 ng/ml). All animals were allowed free access to normal laboratory chow and water.

Serum glucose and insulin levels were determined by Enzymatic Method (Mutaratase-GOD) and ELISA (mouse insulin assay kit) from orbital sinus blood samples obtained before (pretreatment) and 90 minutes after the last vehicle and/or test substance administration (postreatment) and percent change was determined.

Exenatide, a GLP-1 incretin mimetic currently approved for the treatment of type 2 diabetes, is known to increase insulin levels, decrease glucose levels, and decrease body weight. Such a profile is considered beneficial for the treatment of diabetes.

FIG. 1 shows the percentage increase in insulin levels after oral administration of test compounds at 100 and 200 mg/kg and metformin at 300 mg/kg. While metformin does not show an increase in insulin levels in this model, compounds 6a, 12a, and 12b each show significant increases when dosed at 200 mg/kg. Compound 12b shows a significant increase when dosed at 100 mg/kg as well.

FIG. 2 shows the percentage decrease in glucose levels after oral administration of test compounds at 100 and 200 mg/kg and metformin at 300 mg/kg. Compounds 6a, 12a, and 12b each show significant decreases in glucose levels when dosed at 200 mg/kg. The positive control, metformin, also shows a significant decrease in glucose levels when dosed at 300 mg/kg.

FIG. 3 shows the percentage decrease in weight after oral administration of test compounds at 200 mg/kg and metformin at 300 mg/kg. Compounds 6a, 12a, and 12b each show significant decreases in weight when dosed at 200 mg/kg. The positive control, metformin, does not show a decrease in weight when dosed at 300 mg/kg.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula I or a pharmaceutically acceptable salt thereof, or a solvate of the compound or its salt: 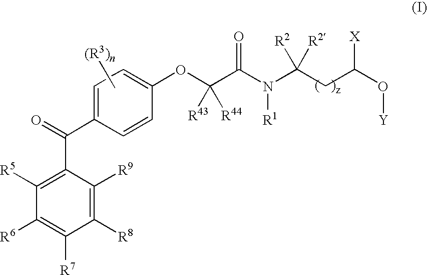
wherein:
X represents H, methyl, or an optionally substituted aryl or heteroaryl ring system;
Y represents an optionally substituted aryl or heteroaryl ring system;
n represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³ independently for each occurrence, represents a substituent;
R⁵, R⁶, R⁷, R⁸, and R⁹ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system;
wherein the compound is optionally characterized by one or more of the following:
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents and optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R⁵, R⁶, R⁷, R⁸, and R⁹ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R⁵, R⁶, R⁸, and R⁹ are hydrogen, and R⁷ is halogen;
R⁷ is chloro;
R³ independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
n is 0; and
R⁴³ and R⁴⁴ each represent hydrogen an optionally substituted lower alkyl.

2-14. (canceled)

15. A compound of claim 1, wherein the compound is a compound of formula Ia or a pharmaceutically acceptable salt thereof, or a solvate of the compound or its salt: 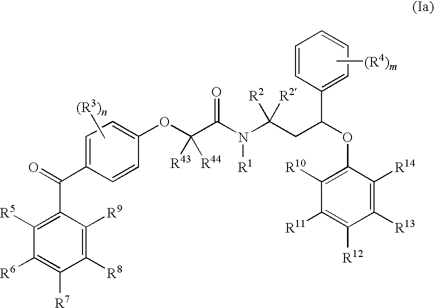
wherein:
n represents an integer from 0 to 4;
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³ and R⁴, independently for each occurrence, represent a substituent;
R⁵, R⁶, R⁷, R⁸, R⁹, R¹⁰, R¹¹, R¹², R¹³, and R¹⁴ each independently represent hydrogen or a substituent; and
R⁴³ and R⁴⁴ each independently represent hydrogen or an optionally substituted lower alkyl, or R⁴³ and R⁴⁴ taken together with the carbon to which they are attached form a three- to six-membered cyclic ring system;
wherein the compound is optionally characterized by one or more of the following:
R⁵, R⁶, R⁷, R⁸, R⁹, R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R⁵, R⁶, R⁸, R⁹, R¹⁰, R¹¹, R¹³, and R¹⁴ are hydrogen, R⁷ is halogen, and R¹² is optionally substituted lower alkyl;
R⁷ is chloro and R¹² is trifluoromethyl;
R³ and R⁴ each independently for each occurrence represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
m is 0;
n is 0;
R⁴³ and R⁴⁴ each represent hydrogen an optionally substituted lower alkyl; and
the compound has the structure 1, 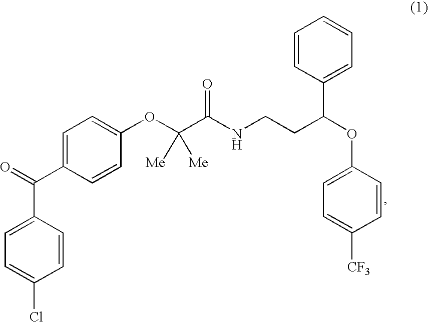
wherein the compound is optionally enriched for the (R) enantiomer.

16-27. (canceled)

28. A compound of formula II or a pharmaceutically acceptable salt thereof, or a solvate of the compound or its salt: 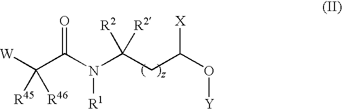
wherein:
X represents H, methyl, or an optionally substituted aryl or heteroaryl ring system;
Y represents an optionally substituted aryl or heteroaryl ring system;
W represents 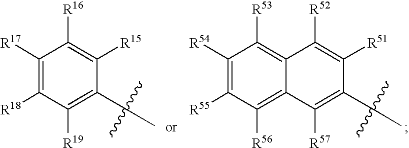
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system;
wherein the compound is optionally characterized by one or more of the following:
X represents an optionally substituted aryl or heteroaryl ring system;
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents an optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, optionally substituted amido, optionally substituted acylamino, optionally substituted heterocyclyl, optionally substituted heterocyclylalkyl, optionally substituted aryl, cyano, or nitro;
R¹⁵, R¹⁶, R¹⁸, and R¹⁹ are hydrogen, and R¹⁷ is optionally substituted lower alkyl;
R¹⁷ is isobutyl;
R¹ represents hydrogen;
R² and R^{2′} are each hydrogen;
R² is hydrogen and R^{2′} is lower alkyl; and
R⁴⁵ is hydrogen and R⁴⁶ is an optionally substituted lower alkyl.

29-40. (canceled)

41. A compound of claim 28, wherein the compound is a compound of formula IIa or a pharmaceutically acceptable salt thereof, or a solvate of the compound or its salt: 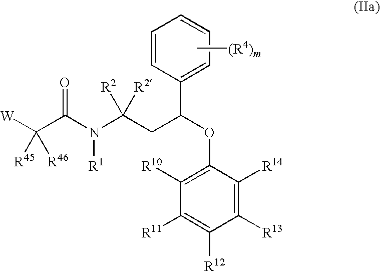
wherein:
W represents 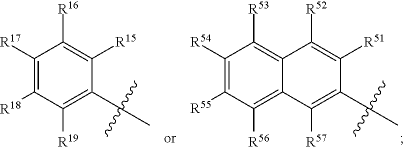
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent;
R¹⁰, R¹¹, R¹², R¹³, R¹⁴, R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen or a substituent; and
R⁴⁵ and R⁴⁶ each independently represent hydrogen or an optionally substituted lower alkyl, or R⁴⁵ and R⁴⁶ taken together with the carbon to which they are attached form a three- to six-membered cyclic ring system;
wherein the compound is optionally characterized by one or more of the following:
R¹⁵, R¹⁶, R¹⁷, R¹⁸, R¹⁹, R⁵¹, R⁵², R⁵³, R⁵⁴, R⁵⁵, R⁵⁶, and R⁵⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, optionally substituted amido, optionally substituted acylamino, optionally substituted carbocyclylalkyl, optionally substituted heterocyclyl, optionally substituted heterocyclylalkyl, optionally substituted aryl, cyano, or nitro;
R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted aminoalkyl, optionally substituted amido, optionally substituted acylamino, optionally substituted heterocyclyl, optionally substituted heterocyclylalkyl, cyano, or nitro;
R¹⁰, R¹¹, R¹³, R¹⁴, R¹⁵, R¹⁶, R¹⁸, and R¹⁹ are hydrogen, and R¹² and R¹⁷ are each optionally substituted lower alkyl;
R¹⁷ is isobutyl and R¹² is trifluoromethyl;
R⁴ independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
m is 0;
R⁴⁵ is hydrogen and R⁴⁶ is an optionally substituted lower alkyl; and
the compound has the structure 4, 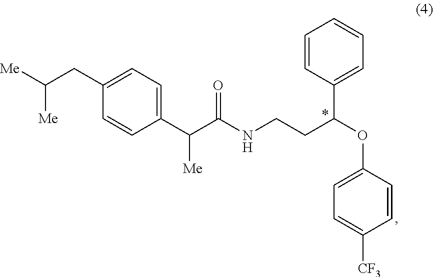
wherein the compound is optionally enriched for the (R) configuration at the indicated position (*).

42-53. (canceled)

54. A compound of formula III or a pharmaceutically acceptable salt thereof, or a solvate of the compound or its salt: 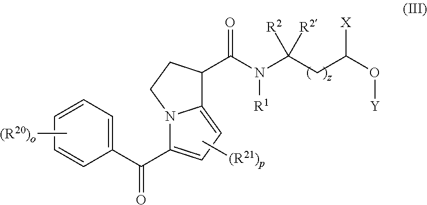
wherein:
X represents H or an optionally substituted aryl or heteroaryl ring system;
Y represents an optionally substituted aryl or heteroaryl ring system;
o represents an integer from 0 to 5;
p represents an integer from 0 to 2;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system; and
R²⁰ and R²¹ each independently for each occurrence represent a substituent;
wherein the compound is optionally characterized by one or more of the following:
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents an optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R²⁰ and R²¹ each independently represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
o is 0;
p is 0;
R¹ represents hydrogen;
R² and R^{2′} are each hydrogen; and
R² is hydrogen and R^{2′} is lower alkyl.

55-64. (canceled)

65. A compound of claim 54, wherein the compound is a compound of formula IIIa or a pharmaceutically acceptable salt thereof, or a solvate of the compound or its salt: 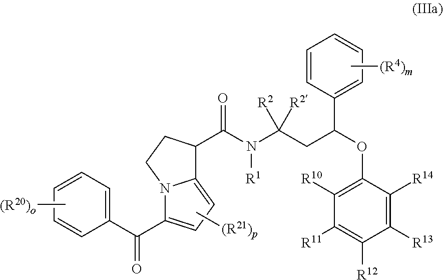
wherein:
m represents an integer from 0 to 5;
o represents an integer from 0 to 5;
p represents an integer from 0 to 2;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²⁰, and R²¹, each independently for each occurrence, represents a substituent; and
R¹⁰, R¹¹, R¹², R¹³, and R¹⁴ each independently represent hydrogen or a substituent;
wherein the compound is optionally characterized by one or more of the following:
R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro.
R¹⁰, R¹¹, R¹³, and R¹⁴ are hydrogen, and R¹² is optionally substituted lower alkyl.
R¹² is trifluoromethyl;
R⁴, R²⁰, and R²¹ each independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
m is 0;
o is 0;
p is 0; and
the compound has the structure 5, 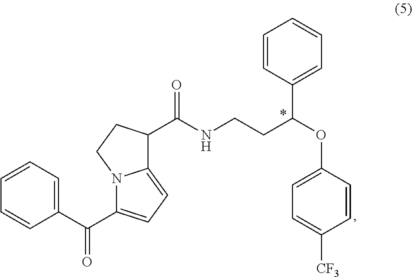
wherein the compound is optionally enriched for the (R) configuration at the indicated position (*).

66-77. (canceled)

78. A compound of formula IV or a pharmaceutically acceptable salt thereof, or a solvate of the compound or its salt: 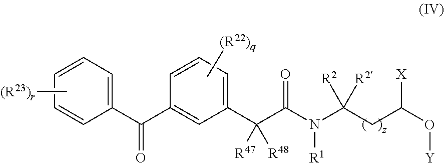
wherein:
X represents H or an optionally substituted aryl or heteroaryl ring system;
Y represents an optionally substituted aryl or heteroaryl ring system;
r represents an integer from 0 to 5;
q represents an integer from 0 to 4;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²² and R²³ each independently for each occurrence represent a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system;
wherein the compound is optionally characterized by one or more of the following:
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents an optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R²² and R²³ each independently represent optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
q is 0;
r is 0;
R¹ represents hydrogen;
R² and R^{2′} are each hydrogen;
R² is hydrogen and R^{2′} is lower alkyl; and
R⁴⁷ is hydrogen and R⁴⁸ is an optionally substituted lower alkyl.

79-89. (canceled)

90. A compound of claim 78, wherein the compound is a compound of formula IVa or a pharmaceutically acceptable salt thereof, or a solvate of the compound or its salt: 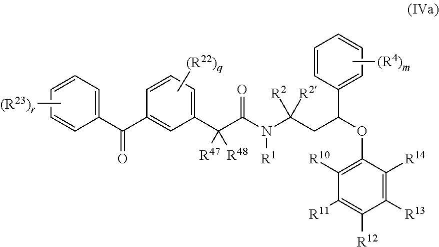
wherein:
m represents an integer from 0 to 5;
r represents an integer from 0 to 5;
q represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, R²², and R²³, each independently for each occurrence, represents a substituent;
R¹⁰, R¹¹, R¹², R¹³, and R¹⁴ each independently represent hydrogen or a substituent; and
R⁴⁷ and R⁴⁸ each independently represent hydrogen or an optionally substituted lower alkyl, or R⁴⁷ and R⁴⁸ taken together with the carbon to which they are attached form a three- to six-membered cyclic ring system;
wherein the compound is optionally characterized by one or more of the following:
R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹⁰, R¹¹, R¹³, and R¹⁴ are hydrogen, and R¹² is optionally substituted lower alkyl;
R¹² is trifluoromethyl;
R⁴, R²², and R²³ each independently for each occurrence represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
m is 0;
q is 0;
r is 0;
R⁴⁷ is hydrogen and R⁴⁸ is an optionally substituted lower alkyl; and
the compound has the structure 6, 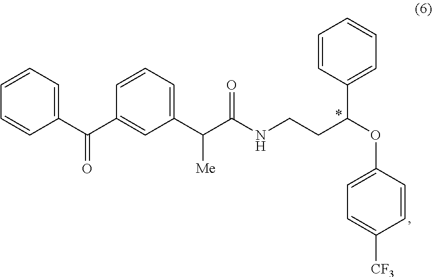
wherein the compound is optionally enriched for the (R) configuration at the indicated position (*).

91-103. (canceled)

104. A compound of formula V or a pharmaceutically acceptable salt thereof, or a solvate of the compound or its salt: 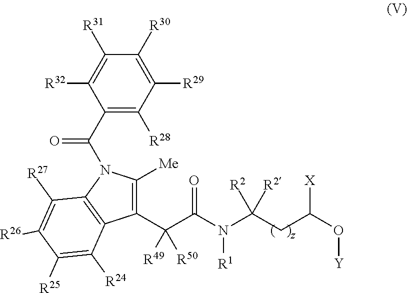
wherein:
X represents H or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system;
wherein the compound is optionally characterized by one or more of the following:
when z is 1, X represents an optionally substituted aryl or heteroaryl ring system;
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents an optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R²⁴, R²⁵, R²⁶, R²⁷, R²⁸, R²⁹, R³⁰, R³¹, and R³² each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R²⁴, R²⁶, R²⁷, R²⁸, R²⁹, R³¹, and R³² each independently represent hydrogen, R²⁵ represents optionally substituted alkoxyl, and R³⁰ represents halogen;
R²⁵ represents methoxy, and R³⁰ represents chloro;
R¹ represents hydrogen;
R² and R^{2′} are each hydrogen;
R² is hydrogen and R^{2′} is lower alkyl; and
R⁴⁹ and R⁵⁰ each represent hydrogen.

105-116. (canceled)

117. A compound of claim 104, wherein the compound is a compound of formula Va or a pharmaceutically acceptable salt thereof, or a solvate of the compound or its salt: 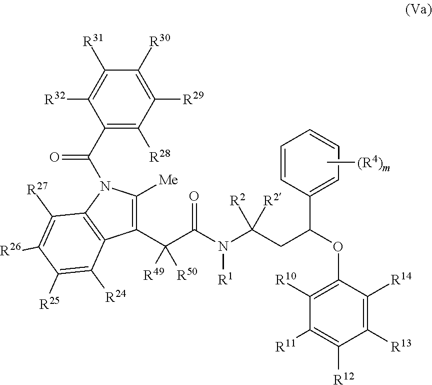
wherein:
m represents an integer from 0 to 5;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R⁴, independently for each occurrence, represents a substituent;
R¹⁰, R¹¹, R¹², R¹³, R¹⁴, R²⁴, R²⁵, R²⁶, R²⁷, R²⁸, R²⁹, R³⁰, R³¹, and R³² each independently represent hydrogen or a substituent; and
R⁴⁹ and R⁵⁰ each independently represent hydrogen or an optionally substituted lower alkyl, or R⁴⁹ and R⁵⁰ taken together with the carbon to which they are attached form a three- to six-membered cyclic ring system;
wherein the compound is optionally characterized by one or more of the following:
R²⁴, R²⁵, R²⁶, R²⁷, R²⁸, R²⁹, R³⁰, R³¹, and R³² each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R²⁴, R²⁶, R²⁷, R²⁸, R²⁹, R³¹, and R³² each independently represent hydrogen, R²⁵ represents optionally substituted alkoxyl, and R³⁰ represents halogen;
R²⁵ represents methoxy, and R³⁰ represents chloro;
R¹⁰, R¹¹, R¹², R¹³, and R¹⁴ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹⁰, R¹¹, R¹³, and R¹⁴ are hydrogen, and R¹² is optionally substituted lower alkyl;
R¹² is trifluoromethyl;
R⁴, independently for each occurrence, represents optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R¹ represents hydrogen;
R² and R^{2′} are each hydrogen;
R² is hydrogen and R^{2′} is lower alkyl;
m is 0;
R⁴⁹ and R⁵⁰ each represent hydrogen; and
the compound has the structure 7, 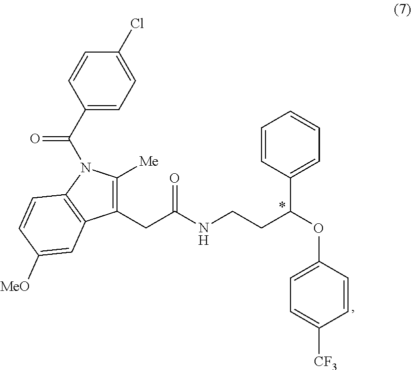
wherein the compound is optionally enriched for the (R) configuration at the indicated position (*).

118-131. (canceled)

132. A compound of formula VI or a pharmaceutically acceptable salt thereof, or a solvate of the compound or its salt: 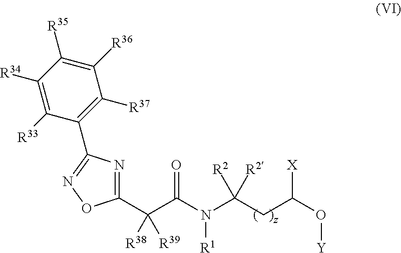
wherein:
X represents H, methyl, or an optionally substituted aryl or heteroaryl ring system;
Y represents an optionally substituted aryl or heteroaryl ring system;
z represents an integer from 0 to 4;
R¹ represents hydrogen or optionally substituted lower alkyl;
R² and R^{2′} each independently represent hydrogen or optionally substituted lower alkyl, or R² and R^{2′} taken together with the carbon to which they are attached form a four- to six-membered cyclic ring system;
R³³, R³⁴, R³⁴, R³⁵, R³⁶, and R³⁷ each independently represent hydrogen or a substituent; and
R³⁸ and R³⁹ each independently represent hydrogen or a substituent, or R³⁸ and R³⁹ taken together with the carbon to which they are attached form a three- to six-membered cyclic ring system;
wherein the compound is optionally characterized by one or more of the following:
X represents an optionally substituted phenyl or thiophene;
X is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
Y represents an optionally substituted phenyl or naphthyl ring system;
Y is optionally substituted with optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R³³, R³⁴, R³⁵, R³⁶, and R³⁷ each independently represent hydrogen, optionally substituted lower alkyl, halogen, hydroxyl, carboxyl, optionally substituted ester, optionally substituted alkoxycarbonyl, optionally substituted acyl, optionally substituted thioester, optionally substituted thioacyl, optionally substituted thioether, optionally substituted alkoxyl, optionally substituted amino, optionally substituted amido, optionally substituted acylamino, cyano, or nitro;
R³³, R³⁴, R³⁶, and R³⁷ are hydrogen, and R³⁵ is optionally substituted lower alkyl;
R¹ represents hydrogen;
R² and R^{2′} are each hydrogen;
R³⁸ and R³⁹ each independently represent hydrogen, halo, optionally substituted lower alkyl, or optionally substituted piperidine;
R³⁸ represents hydrogen and R³⁹ represents 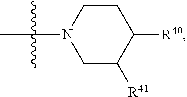
wherein
R⁴⁰ represents hydrogen, optionally substituted lower alkyl, or optionally substituted amino, such as N-methyl-N-benzylamino; and
R⁴¹ represents hydrogen or optionally substituted lower alkyl, such as (4-(trifluoromethyl)phenoxy)methyl;
R³⁸ represents hydrogen and R³⁹ represents 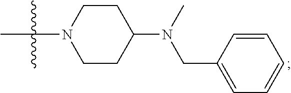
and
R³⁸ represents hydrogen and R³⁹ represents 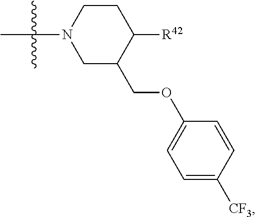
wherein
R⁴² represents hydrogen or optionally substituted lower alkyl.

133-144. (canceled)

145. A pharmaceutical composition comprising a pharmaceutically acceptable carrier and a compound of any of claims 1, 15, 28, 41, 54, 65, 78, 90, 104, 117, or 132.

146. A method of treating depression, obesity, metabolic syndrome, or a disorder associated with metabolic syndrome in a mammal, comprising administering to a mammal suffering from depression, obesity, metabolic syndrome, or a disorder associated with metabolic syndrome, a compound of any of claims 1, 15, 28, 41, 54, 65, 78, 90, 104, 117, or 132;
wherein the method is optionally characterized by one or more of the following:
wherein the disorder associated with metabolic syndrome is selected from obesity, diabetes, hypertension, or hyperlipidemia;
wherein the disorder associated with metabolic syndrome is diabetes; and
wherein said mammal is a human.

147-150. (canceled)
